(19) United States
(12) Patent Application Publication
(10) Pub. No.: US 2025/0346628 A1
(43) Pub. Date: Nov. 13, 2025

(54) PHOTONICALLY ACTIVE BOWTIE NANOASSEMBLIES WITH CHIRALITY CONTINUUM AND APPLICATIONS THEREOF IN MACHINE VISION

(71) Applicant: The Regents of The University of Michigan, Ann Arbor, MI (US)

(72) Inventors: Minjeong CHA, Foster City, CA (US); Prashant KUMAR, Ann Arbor, MI (US); Nicholas A. KOTOV, Ypsilanti, MI (US)

(73) Assignee: The Regents of The University of Michigan, Ann Arbor, MI (US)

(21) Appl. No.: 18/861,031

(22) PCT Filed: Apr. 28, 2023
(86) PCT No.: PCT/US2023/020412

§ 371 Date: Oct. 28, 2024

Related U.S. Application Data
(60) Provisional application No. 63/336,954, filed on Apr. 29, 2022.

(51) Int. Cl. C07K 5/062

(52) U.S. Cl. CPC C07K 5/0606

(57) ABSTRACT

Nanostructured chiral microparticles with bowtie shape having widely variable pitch, size, thickness and length are provided. The self-limited assembly of anisotropic building blocks makes possible high synthetic reproducibility, size monodispersity and computational predictability of their geometries for different assembly conditions. They display multiple strong circular dichroism peaks originating from absorptive and scattering phenomena. Unlike classical chiral molecules, these particles display a continuum of Osipov-Pickup-Dunmur chirality measures that exponentially correlate with the spectral positions of the circular dichroism peaks. Bowtie particles with variable polarization rotation were utilized in printing photonically active metasurfaces with spectrally tunable positive/negative polarization signatures for light detection and ranging (LIDAR) devices.

### CROSS-REFERENCE TO RELATED APPLICATIONS

This application claims the benefit of U.S. Provisional Application No. 63/336,954, filed on Apr. 29, 2022. The disclosure of the above application is incorporated herein by reference in its entirety.

### GOVERNMENT SUPPORT

This invention was made with government support under N000141812876 and HQ0342010033 awarded by the U.S. Office of Naval Research. The Government has certain rights in the invention.

### FIELD

The present disclosure relates to chiral microparticles having a bowtie shape comprising assemblies of nanoribbons that exhibit tailored chirality properties.

### BACKGROUND

This section provides background information related to the present disclosure which is not necessarily prior art.

Certain materials with microscale and/or nanoscale chirality are known to strongly rotate the polarization of linearly polarized (LinP) and circularly polarized light (CPL). Chirality of a microparticle or nanoparticle means that the structure exhibits asymmetrical optical activity with different handedness, for example, clockwise to form left handed chirality (S- or L-orientation) and counter-clockwise to form right handed chirality (R- or D-orientation). Such optical effects with different chiral geometries are being actively investigated as a part of chiral photonics and plasmonics for machine vision and the like. Chirality is a geometrical property described by continuous mathematical functions. However, chirality is often treated in chemical disciplines as binary left/right characteristic of molecules rather than a continuity of chiral shapes. While being theoretically possible, a family of stable chemical structures with the same shape and progressively tunable chirality is not yet known.

It would be desirable to provide nanostructured microparticles providing a high degree of control over chirality by providing variable size, pitch, thickness, length, and the like.

### SUMMARY

This section provides a general summary of the disclosure, and is not a comprehensive disclosure of its full scope or all of its features.

In certain aspects, the present disclosure contemplates a chiral microparticle that may comprise an assembly comprising a plurality of nanoribbons that defines a bowtie shape and exhibits chirality.

In one aspect, each nanoribbon of the plurality of nanoribbons comprises at least two peptides interconnected by at least one cadmium ion (Cd²⁺).

In one aspect, each nanoribbon of the plurality of nanoribbons comprises at least two cystine molecules interconnected by at least one cadmium ion (Cd²⁺).

In one aspect, each nanoribbon of the plurality of nanoribbons comprises at least two homocystine molecules interconnected by at least one cadmium ion (Cd²⁺).

In one aspect, each nanoribbon of the plurality of nanoribbons has a length of greater than or equal to about 10 nm to less than or equal to about 100 micrometers.

In one aspect, each nanoribbon of the plurality of nanoribbons has a thickness of greater than or equal to about 50 nm to less than or equal to about 10 micrometers.

In one aspect, each nanoribbon of the plurality of nanoribbons has a width of greater than or equal to about 3 nm to less than or equal to about 20 micrometers.

In one aspect, the chirality is within an OPD index of −150 to +150.

In one aspect, the assembly defines the chiral microparticle having a length of greater than or equal to about 100 nm to less than or equal to about 100 micrometers.

In one aspect, the assembly defines the chiral microparticle having a thickness of greater than or equal to about 500 nm to less than or equal to about 10 micrometers.

In one aspect, the assembly defines the chiral microparticle having a width of greater than or equal to about 50 nm to less than or equal to about 30 micrometers.

In one aspect, the assembly defines the chiral microparticle having a pitch of greater than or equal to about 10 nm to less than or equal to about 100 micrometers.

In certain other aspects, the present disclosure contemplates a chiral dispersion that comprises a plurality of chiral microparticles each comprising a plurality of nanoribbons distributed in a medium. At least one first chiral microparticle in the plurality of chiral microparticles exhibits a first chirality that is distinct from a second chirality exhibited by at least one second chiral microparticle in the plurality of chiral microparticles.

In one aspect, the plurality of nanoribbons each comprises at least two peptides interconnected by at least one cadmium ion (Cd²⁺).

In one aspect, the plurality of nanoribbons each comprises at least two cystine molecules interconnected by at least one cadmium ion (Cd²⁺).

In one aspect, the plurality of nanoribbons each comprises at least two homocystine molecules interconnected by at least one cadmium ion (Cd²⁺).

In one aspect, the plurality of nanoribbons each has a length of greater than or equal to about 10 nm to less than or equal to about 100 micrometers, a thickness of greater than or equal to about 50 nm to less than or equal to about 10 micrometers, and a width of greater than or equal to about 3 nm to less than or equal to about 20 micrometers.

In one aspect, the first chirality is within an OPD index of −150 to +150.

In one aspect, the at least one first chiral microparticle has a length of greater than or equal to about 100 nm to less than or equal to about 100 micrometers, a thickness of greater than or equal to about 500 nm to less than or equal to about 10 micrometers, a width of greater than or equal to about 50 nm to less than or equal to about 30 micrometers, and a pitch of greater than or equal to about 10 nm to less than or equal to about 100 micrometers.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1K. SEM images of bowties formed from Cd²⁺ and FIG. 1A, L-CST (left), FIG. 1B, rac-CST (middle), and FIG. 1C, D-CST (right). Scale bars are 2 μm. Circular dichroism (top) and extinction spectra (bottom) in FIG. 1D, THz, FIG. 1E, mid-IR (VCD) and FIG. 1F, UV-vis-NIR ranges for bowtie particles. FIG. 1G, Zeta potential of fully formed bowties at different ratios of L-CST and Cd²⁺ ions. FIG. 1H, Zeta potential of bowties formed from L-CST solution at different pH. FIG. 1I, Synchrotron XRD pattern and the FIGS. 1J-1K, calculated crystal structure of nanosheets and nanoplatelets forming the bowties. Unit cell is shown in FIG. 9.

FIGS. 2A-2J show non-binary evolution of chirality at micrometer scale. FIG. 2A, SEM image of a fully formed bowtie, composed of twisted segments that are assembled from nanoribbons (FIG. 2B). FIG. 2C, SEM image of nanoribbons composed of polydisperse nanoplatelets. FIG. 2D, TEM image of the platelet. FIG. 2E, cryo-SAED pattern of the nanosheet in FIG. 2D. FIG. 2F, SEM images of the bowtie particles obtained by mixing different ratios of L-CST and D-CST, as defined by the enantiomeric excess (χ) show the transition from left-handed to pancake to right-handed bowties. Coarse grained particle shape deduced from the atomic structure of the cluster with the hydrogen bonding interaction sites marked by the spheres in LCL and LCD type clusters are shown. Potentials of mean force obtained for preferred directions of interaction between two basic clusters of two permutations of chirality in CST ligands: FIG. 2G, LCL-LCL and FIG. 2H, LCD-LCD are computed and used for MC growth simulations. FIG. 2I, Magnified snapshots from MC simulations of the fully formed petal shows mismatched domains with local crystalline order arranged to form a (FIG. 2J-top) twisted nanoribbon and (FIG. 2J-bottom) pancake type petal for LCL and LCD type clusters, respectively.

FIGS. 3A-3E show morphological diversity of bowties. FIG. 3A, Effect of NaCl, pH, excess charge (L-CST/Cd), solvent (1:1 water to solvent) on bowtie morphology (water, [Cd²⁺]=1 mM, 4 mM for pH, [L-CST]=4 mM) are shown. Scale bar in FIG. 3A is 1 μm. FIG. 3B, Continuously variable twist and sizes for bowtie particles obtained for different χ and [Cd²⁺]. Scale bar in FIG. 3B is 5 μm. FIG. 3C, SEM image of a typical bowtie assembly and the corresponding morphological parameters of length (l), width (w), thickness (t) and pitch (2w tan (90−θ)) overlaid. Three-dimensional model (right) constructed using the aforementioned parameters to compute the OPD indices for estimation of the structural chirality. FIG. 3D, Variability of the geometrical parameters for the bowtie assemblies obtained in this study and the same parameters obtained in other studies. FIG. 3E, Variation of the morphological parameters with OPD chirality measure.

FIGS. 4A-4G show optical properties of the bowtie particles. FIG. 4A, Normalized CD spectra for bowties of different OPD with peaks P1 (−), P2 (+), P3 (−) labeled and the FIG. 4B, corresponding extinction spectra, FIG. 4C, Variation of peaks P1, P2, P3 with chirality parameter for bowties made with varying [L-CST]/[Cd²⁺]. Models of left-handed twisted single sheets were constructed with sizes of 250 nm-1000 nm and their FIG. 4D, simulated g-factor spectra and e, extinction spectra are shown here. FIG. 4F, Projections of 1 μm long sheet with a left-handed twist used as models for calculation of optical properties. Corresponding scattered electric field of 2.5 (V/m)² are overlaid when the model interacts with LCP (red) and RCP (blue) of wavelength 1100 nm and 385 nm. The electrical field around the bowtie model twists stronger when the handedness of photons and the particles is the same. FIG. 4G, Photograph of L-shaped coatings deposited on glass are illuminated with a 1550 nm LIDAR laser and the backscattered signal is shown under it.

FIGS. 5A-5B show self-assembly of bowtie particles. FIG. 5A, Photographs of the change in turbidity of the [L-CST]=1 mM solution upon addition of [Cd²⁺]=1 mM ions as time increases. The rate of change in turbidity is proportional to the [Cd²⁺] where higher concentration of metal ion causes a faster change in turbidity. FIG. 5B, CD spectra of bowtie particle dispersions acquired during their formation.

FIGS. 6A-6B. Vibrational spectroscopy of the bowtie particles. FIG. 6A, Raman spectra from two different bowtie morphologies assembled with different starting [L-CST] and [Cd²⁺]. Both synthetic conditions result in different macroscale morphology but same Raman spectra indicating that the molecular structure of smallest building blocks are same. FIG. 6B, FTIR spectra of L-cysteine (free amino acid, not cystine), L-CST, bowties from L-CST and D-CST in powder form. Note: Presence of S—S disulfide stretches in Raman spectra around 500 cm⁻¹ is a confirmation that dipeptide bond is not broken in the self-assembled structure. As compared to L-CST bowties have a weak broad shoulder peak in 3000-3750 cm⁻¹ range with sharp peaks on top of it, indicating the presence of directional hydrogen bonding. Simulated vibrational configurations of a Cystine molecule at which the Cd₂CST₂ bowties demonstrate chiral fingerprint in VCD spectrum. Note the —NH₂ scissoring inwards (1593 cm⁻¹) and outwards (1557 cm⁻¹) are the most intense in the VCD spectra.

FIG. 7. Simulated vibrational configurations of a Cystine molecule at which the Cd₂CST₂ bowties demonstrate chiral fingerprint in VCD spectrum. Note the —NH₂ scissoring inwards (1593 cm⁻¹) and outwards (1557 cm⁻¹) are the most intense in the VCD spectra. Atomic structure of the atomic unit in the constituent nanocluster, comprising of L-CST molecule, Cd²⁺ used for the simulated annealing procedure in structure solution.

FIG. 8. Synchrotron XRD data for freeze-dried powders of Cd₂(L-CST)₂ (red), Cd₂(D-CST)₂ (blue) and Cd₂(DL-CST)₂ (black) bowties are plotted.

FIG. 9. Three perpendicular projections of the atomic structure of the refined structure of Cd₂(L-CST)₂ unit cell. Atoms are represented as Oxygen (red), Carbon (black), Cadmium (Brown), Nitrogen (blue), Sulfur (yellow).

FIG. 10 is a table showing experimental details.

FIG. 11. Three perpendicular projections of the atomic structure of the refined structure of Cd₂(L-CST)₂ unit cell. Atoms are represented as Oxygen (red), Carbon (black), Cadmium (Brown), Nitrogen (blue), Sulfur (yellow).

FIGS. 12A-12C. FIG. 12A, Comparison between the observed (blue) and calculated XRD spectra (red) obtained from the solved structure. FIG. 12B, Atomic structure of 4×2×2 Cd₂(L-CST)₂ supercell of bow-ties is shown along [001], [010], and [100] zone axis. FIG. 12C, Side view of the supercell with the layer of Cd—O is extended out for clarity. The top view of the Cd—O layer is shown on the right with the helical chains are highlighted with spherical markers.

FIGS. 13A-13F. Electron dose sensitivity of bowties. FIG. 13A-13B, Annular dark field scanning transmission electron microscopy (ADF-STEM) image and FIGS. 13C-13D, corresponding electron diffraction pattern (right) for bowties at room temperature (300 K) and cryogenic temperature (below 128 K). At room temperature the Cd-CST bowties convert into CdS nanoparticles as evidenced by emergence of the broad peaks at 2.9 nm⁻¹ and 4.8 nm⁻¹ characteristic of CdS. FIG. 13E, Line spectra of the rotationally averaged electron diffraction pattern shown in panels FIG. 13C and FIG. 13D at 300 K (blue) and <128 K (orange). FIG. 13F, Electron diffraction pattern obtained at <128 K (blue) and synchrotron XRD pattern (orange) confirming the transition of bowties into nanoparticles under the electron beam at room temperature. The primary peak in XRD pattern corresponding to inter-chain distance at 10.2 Å is blocked by the beam stopper in SAED pattern.

FIGS. 14A-14E. Conversion of Cd-CST bowties into CdS bowties. FIG. 14A, ADF-STEM image and FIG. 14B, Electron diffraction pattern of bowties. FIGS. 14C, 14D, High-resolution ADF-STEM image of bowtie shown in a, confirming the presence of 2-5 nm sized NPs of CdS. FIG. 14E, Rotationally averaged electron diffraction (blue) shown in panel b, with a modeled electron diffraction for 2.5 nm sized NPs (orange). The corresponding peaks of CdS from crystallographic database for PDF 10-0454.

FIGS. 15A-15B. Coarse Grained model of the Cd-CST nanoclusters. FIG. 15A: (Left) XRD resolved structure of building block. (Middle) Convex hull around atoms. (Right) Patchy model of convex hull of the nanocluster. FIG. 15B, Construction of generalized building block. Relevant features such as sites for hydrogen bonding (H-bonding), charge-charge repulsion, and sulfur linkage sites are placed on the generalized building block to mimic their general location on the XRD resolved patchy convex hull of the nanocluster in FIG. 15A).

FIGS. 16A-16B. Monodispersity of the self-assembled bowties. SEM images of the FIG. 16A, Cd₂(L-CST)₂ and FIG. 16B, Cd₂(D-CST)₂ bowties formed from [L-CST] and [Cd²⁺] as 4 mM in 200 ml water.

FIGS. 17A-17B. ZZ6 Monte-Carlo simulation of the growth of nanoribbons and their stacks. Snapshots of particle growth simulations for FIG. 17A, χ=0 and FIG. 17B, χ=1. Ordered domains are visible in both growth conditions. Colors of the individual units represent the normalized time at which the nanoribbons attach to the existing bowtie particle.

FIGS. 18A-18C. STEM-EDX based evaluation of atomic distribution. FIG. 18A, Spatially resolved energy dispersive X-ray spectroscopy for the bowties made with different [Cd²⁺], [L-CST] concentrations. Atomic concentration of S (green) and Cd (Red) was in the ratio 2:1, indicative of one CST molecule sharing one Cd atom. This ratio was used to construct a Cd-CST fragment as an input for atomic structure solution from 3D electron density maps obtained from powder XRD data. The presences of extra Cd²⁺ at the interface indicates positive surface charge. FIG. 18B, Cross-sectional analysis of the bowtie at the center node was performed and revealed the S/Cd ratio of 1.67, which could occur due to transformation by Ga-ion beam during thinning or due to a difference in the core composition. FIG. 18C, High-resolution ADF-STEM images of the core of a right-handed bowtie indicate a fibrous structure made of polydisperse NPs.

FIGS. 19A-19C. High magnification SEM images of bowtie particles. Shown are the SEM images of FIG. 19A, fully formed bowtie, FIG. 19B, magnified region at the center of bowtie and FIG. 19C, magnified region at the edges of the bowtie. Synthesis conditions are [Cd²⁺]=4 mM, [L-CST]=4 mM, Water=200 ml. Polydisperse nanoplatelets with diameters between 20-100 nm are assembled into nanoribbons. In turn, they are stacked into the bowties with the left twist. Smaller nanoplatelets lead to stronger twisting.

FIGS. 20A-20C. Geometry of the bowtie particles made with different L-CST/Cd ratios. FIG. 20A: Geometrical parameters characterizing length, width and thickness of the bowties, namely I, w, and t depicted in FIG. 3C, increase with the increase of [L-CST]/[Cd²⁺] ratio. FIG. 20B, Zeta potentials (red) of as-made bowtie particles made with different [L-CST]/[Cd²⁺] ratios in the original synthetic media increases with crossover at 0 potential occurring near [L-CST]/[Cd²⁺]=1. FIG. 20C, Zeta potentials of bowtie particles made with different [L-CST]/[Cd²⁺] ratios after centrifugation and re-dispersion in DI water. Comparison with the zeta-potentials in original reaction media indicate that the charge of the bowtie surface is associate with adsorption of excess ions of L-CST or Cd²⁺ depending on the [L-CST]/[Cd²⁺] ratios. The excess positive and negative charge is depleted upon centrifugation. Note: The dependence l, w, and t of the fully assembled particles is consistent with the dependence of thickness and width of individual nanoribbons on [L-CST] and L-CST/Cd ratio (see also FIGS. 24A-24C, 30A-30D).

FIG. 21. Change in pH of the solution as the ratio of [L-CST]/[Cd²⁺] is changed.

FIGS. 22A-22B. Role of pH in self-assembly. FIG. 22A, Variation of zeta-potential of fully formed particles with increasing pH of [L-CST]=4 mM solution before addition of [Cd²⁺]=4 mM. pH was controlled by adding NaOH in the range of 0.7 to 1 ml volume to a 10 ml solution of L-CST. Cystine is typically soluble above pH 8 and below pH 2. FIG. 22B, SEM images of the self-assembled bowties after addition of Cd²⁺ ions to the corresponding L-CST solution. It is notable that the twisting of the self-assembly begins above pH 12, which also coincides with the negative zeta potential of the bowties. Scale bar across all images in 1 μm.

FIGS. 23A-23C. Quantification of the consumed L-CST during the bowtie synthesis. FIG. 23A, Absorbance of supernatant from reactions of [Cd²⁺]=2 mM with varying [L-CST] (0.5 mM to 10 mM). FIG. 23B, Calibration chart for absorbance of [L-CST] vs absorbance at 250 nm is shown here with black circles representing absorbance from only [L-CST] and red-circles representing estimating [L-CST] in supernatant. FIG. 23C, [L-CST] vs [L-CST] added for samples shown in panel a. Up to 20 μl or (2 mM) [L-CST], all added [L-CST] is consumed (blue curve) and the curve is linear. Beyond 20 μl added [L-CST], 21.7±2.9 μl is consumed, indicating that the [Cd²⁺] and [L-CST] interact in 1:1 ratio to precipitate the bowties.

FIGS. 24A-24C. Geometry of the nanoribbons for various synthetic conditions. SEM image of the ribbons FIG. 24A, facing the electron beam and FIG. 24B, resting on the silicon wafer in edge-on orientation. FIG. 24C, Tabulated width and thickness for changing [L-CST]/[Cd²⁺] ratio. As [L-CST] and [L-CST]/[Cd²⁺] ratio decreases, the width and thickness of the nanoribbons also decrease. Note: The correlation with [L-CST] and [L-CST]/[Cd] ratio is fully consistent with that of the length, width and thickness of the entire assembled particles (FIGS. 30A-30D). Increasing the concentration of ions in the system for CST and Cd²⁺ is not the same. If the CST is increased, the number of —COO and —NH₂ groups are increasing, which simply extends the long range order over several length scales. However, increasing the Cd²⁺ ion concentration changes the platelet charge to positive because they get adsorbed on the negatively charged surface. This creates a repulsion between positively charged platelets but they cannot form hydrogen bonds over long range, because carbonyl groups are limited and therefore the overall size of the bowtie decreases. In the figure above as [L-CST]/[Cd²⁺] ratio increases that means relative concentration of [Cd²⁺] ions are decreasing in number, so the sheets should be negatively charged.

FIG. 25. Schematic of the different physicochemical parameters used to exert structural control over the bowtie assemblies. Changing one or two parameters (such as temperature, EE, counter-ions, solvents, and NaCl) with varying [Cd²⁺] and [L-CST] results in a wide range of continuously variable bowtie assemblies.

FIG. 26. Phase diagram for the combinations of [L-CST] and [Cd²⁺] concentrations varying from 0.6 mM to 4 mM. Increase in [L-CST] with [Cd²⁺] constant (along the columns) results in increase in size of bowties. Increase in [Cd²⁺] while keeping [L-CST] constant (along the rows) results in decrease in size of bowties. Scale bar is 5 μm.

FIG. 27. g-factor spectra from UV-vis to NIR region is plotted for fixed [L-CST] concentration corresponding to each row in phase diagram shown in FIG. 26. As the [L-CST] concentration increases, the variations in g-factor spectra are amplified. From 800-1200 nm the detector changes resulting in a jump in the spectra due to gain correction differences between two detectors.

FIG. 28. Normalized extinction spectra is plotted for fixed [L-CST] concentration corresponding to each row in phase diagram shown in FIG. 26. As the [L-CST] concentration increases, the extinction peak red-shifts. Some extinction spectra have a lower signal to noise ratio because of lower particle concentration of larger sample in 1 ml vial.

FIG. 29. Phase diagram for the combinations of [L-CST] and [Cd²⁺] concentrations varying from 0.6 mM to 4 mM in the presence of added 0.25 M NaCl in water. Increase in [L-CST] with [Cd²⁺] constant (along the columns) results in increase in size of bowties. Increase in [Cd²⁺] while keeping [L-CST] constant (along the rows) results in decrease in size of bowties. Scale bar is 5 μm.

FIGS. 30A-30D. Measurements of Length (l) (FIG. 30A), Width (w) (FIG. 30B), Thickness (t) (FIG. 30C) and Twist Angle (θ) (FIG. 30D) of the stack of twisted sheets are plotted with respect to the ratio of [L-CST]/[Cd²⁺]. Red dots indicate the synthesis conditions without NaCl and blue dots are synthesized in the presence of 0.25 M NaCl. Error bars are calculated from 5-10 measurements of the respective quantity. Note the larger relative increase in thickness for bowties made in 0.25 M NaCl as compared to 0 M NaCl.

FIGS. 31A-31B. Comparison of theory vs Monte Carlo simulations for incomplete cluster formation. FIG. 31A, Twist angle and FIG. 31B, Length of bowties. Solid lines are theory predictions. Scatter points are MC growth simulation results.

FIGS. 32A-32B. Temperature effect on the morphology of Cd₂CST₂ based nanostructured self-assembled particles. FIG. 32A, SEM micrographs of the final bowtie morphology when temperature is varied (along y-axis) from 20-80° C. and [L-CST]/[Cd²⁺] ratio is decreased from 8 to 0.2 ([L-CST]=0.8 mM). FIG. 32B, The morphologies are classified based on their curvature and stacking patterns. Tracing the y-axis from 20° C. to 80° C. at a fixed [L-CST]/[Cd²⁺] ratio indicates that the change in stacking and overall sizes. Scale bar in a is 5 μm.

FIGS. 33A-33E. Effect of changing the enantiomeric excess. FIG. 33A, SEM images of bowties as enantiomeric excess (χ) defined as ([L-CST]−[D-CST])/([L-CST]+[D-CST]) is increased from −1 to 1. FIG. 33B, corresponding extinction and FIG. 33C, g-factor spectra for the morphologies shown above are plotted. FIG. 33D, measured morphology parameters of length, width, thickness and FIG. 33E, twist angle are plotted. The continuous change of twist angle coupled with a parabolic change in length, width and thickness is a clear indication of the competing mechanical, electrostatic and chemical energy penalties continuously changing with χ.

FIGS. 34A-34C. FIG. 34A, LCL and FIG. 34B, LCD building blocks. L is the characteristic length of the monomeric building block nanocluster along its long axis, α is the aspect ratio measuring the amount of nanocluster building block elongation, n is the number of nanoclusters in the assembly, and γ₁ and γ₂ are the local curvature of the nanoclusters along the long and short direction, respectively. FIG. 34C, Schematic of configurations considered in derivation of Aₙ₁ and Aₙ₂. The 2⁻¹ factor accounts for symmetry of the Aₙ₂ configuration being the same going in the 3^{rd} direction (in/out of page).

FIGS. 35A-35B. Comparison of Various Bowtie Dimensions. Comparison of theory, Monte Carlo growth simulation and experiments for FIG. 35A) twist angle and FIG. 35B) various bowtie dimensions.

FIG. 36. Self-Limited Assembly of Bowties. Theoretical prediction of self-limiting sizes (in scaling units) of pancake and bowtie particles as a function number of number of nanoclusters in the particle. Plateau indicates the self-limiting size R* of the growing particles. Dashed lines indicate the self-limiting number of nanoclusters in the growing particles as predicted from Eq. 11 (i.e. particle stops growing at location of dashed lines).

FIGS. 37A-37E. Chiroptical spectra of bowtie particles with different enantiomeric excess. G-factor spectra for bowties with morphologies shown in FIG. 3B acquired for [Cd²⁺] equal to FIG. 37A, 0.6 mM, FIG. 37B, 1.0 mM, FIG. 37C, 2.0 mM, FIG. 37D, 3.0 mM, and FIG. 37E, 4.0 mM while maintaining [L-CST]=4 mM.

FIGS. 38A-38B. Effect of counter-ions on the hierarchical structure of bowties. FIG. 38A, SEM images indicating the changes in the morphology of bowties as the M+/CST ratio is increased for CdI₂. CdBr₂, CdCl₂, and Cd(CH₃COO)₂ as the starting metal salt. Same amount of twist in bowties is produced by different concentration of the metal salt in the following order [I⁻]>[Br⁻]>[Cl⁻]>[CH₃COO⁻]. This series is in some agreement with the typical Hofmeister series but whether this match is coincidental or significant finding will need to be evaluated further. FIG. 38B, Corresponding g-factor spectra for the morphologies shown above. Scale bar in (FIG. 38A) is 5 μm.

FIGS. 39A-39F. Effect of different metal-ions on macroscale morphology. FIG. 39A, Typical morphology of bowtie is shown in the first row which decreases in size as metal ion (M⁺) concentration increases. Copper shows a cross-stitched assembly of fibers which transitions into long individual fibers upon increasing metal ion concentration. Mg²⁺ and Ni²⁺ result in spherical supraparticles, while Zn²⁺ results in a rod-like morphology. The pH of CST solution was fixed at 11 while metal ion concentration was increased. These results indicate that different metal ions coordinate in a different manner with the same CST at the molecular level, thereby affecting the overall macroscale morphology. FIG. 39B, Photographs of the as prepared samples in centrifuge tubes of 2 ml total volume shown in panel a. Corresponding CD spectra for FIG. 39C, Cd²⁺, FIG. 39D, Cu²⁺, FIG. 39E, Zn²⁺, and FIG. 39F, Mg²⁺ are shown here. Scale bar in a is 5 μm.

FIGS. 40A-40B. Effect of changing the solvent. FIG. 40A, SEM images of bowties self-assembled in 100% by volume of different solvents (x-axis) and the effect of varying [L-CST]/[Cd²⁺] ratio. FIG. 40B, Corresponding CD spectra are shown on the right. Scale bar in a is 10 μm. Note: Solvent affects the interaction between the charged molecules by acting as a screening medium with an effective polarizability and permittivity. This in-turn affects the self-assembly where the transition from stacked bundles in water to individual twisted tapes in ethanol to smaller stacks of twisted bundles in DMF are observed. These morphologies can be further controlled by mixing water and solvent in different ratios.

FIGS. 41A-41B. Effect of mixing different solvent with water. FIG. 41A, SEM images of the bowties self-assembled in a 50:50 mixture of water and solvents. Changing the solvent along x-axis results in a decrease in stacking thickness, while the overall size decreases along the y-axis as amount of [Cd²⁺] is increased. FIG. 41B, Corresponding CD spectra for the morphologies shown in a is plotted from UV-vis to NIR region. Scale bar in a is 10 μm. Note. Water-solvent mixtures provide another handle to control the self-assembly pathway of nanoplatelets by changing the interactions between them.

FIGS. 42A-42B. Effect of changing the surface ligands in the bowties. SEM image of bowtie made from FIG. 42A, L-CST and FIG. 42B, L-homocystine. The twist in the overall morphology increases as the chain length of L-CST molecule is increased by two carbon atoms to L-homocystine. The stick models of corresponding molecules are overlaid. Note that the reversal in handedness of molecules results in reversal in handedness of the bowtie.

FIGS. 43A-43B. Synthesis and optical properties of bowtie particles. FIG. 43A, SEM images of bowtie assemblies synthesized with [Cd²⁺]=2 mM and [L/D-CST]=2-4 mM. As [L-CST]/[Cd²⁺] increases the size of bowtie assemblies increases and correspondingly the FIG. 43B, CD spectra red shifts.

FIG. 44. SEM images and the corresponding OPD indices are shown here for bowties of different sizes for which the g-factor spectra is plotted in FIG. 4A.

FIGS. 45A-45B. Osipov-Pickup-Dunmur chirality measures are plotted for the phase diagrams of FIG. 45A, varying [L-CST] and [Cd²⁺] concentrations. FIG. 45B, Varying enantiomeric excess χ vs. [L-CST]/[Cd²⁺]. Color bars indicate the values of OPDs indices and are positive for panel a and symmetrically opposite in sign for panel b. Note: Empirical relationship between different morphological parameters and OPD chiral index is as follows—

$$l = 0.01366 {( {G}_{o} )}^{3.908} + 1.654 , {R}^{2} = 0.6313$$

$$w = 0. 0009684 {( {G}_{o} )}^{5.167} + 0.8329 , {R}^{2} = 0.6486$$

$$p = 0. 0004143 {( {G}_{o} )}^{5.69} + 2.657 , {R}^{2} = 0.4421$$

$$t = 0. 06352 {( {G}_{o} )}^{1.441} + 0.1806 , {R}^{2} = 0.3183$$

FIGS. 46A-46E. Single particle scattering. FIG. 46A, SEM images and FIG. 46B, the corresponding optical micrographs of bowties of increasing size. FIG. 46C, Scattering intensity spectra upon illumination of bowties with unpolarized light obtained from averaging data from 10 single particles of same size. FIG. 46D, Simulated real (n) and imaginary part (k) of refractive index for CdS and Cd₂(L-CST)₂. FIG. 46E, Simulated scattering cross-section for bowties of increasing sizes obtained as averages of polarized light incident in parallel and perpendicular orientation. The scattering intensity increases as the sizes of bowties increase. Overall, the scattering intensity is increased in the NIR regions as compared to visible region upon increase in size of bowties. Above trend is also observed upon simulations of bowties of different sizes.

FIGS. 47A-47D. Calculated optical properties of bowties with geometry as l-1000 nm, w-500 nm, t-50 nm, p-2000 nm. FIG. 47A, CD spectra and FIG. 47B, g-factor of bowties. FIG. 47C, Differential scattering and absorbance contributions to the CD spectra of left-handed bowties. FIG. 47D, Extinction cross-section for a left-handed bowtie interacting with LCP and RCP. All the spectra are an average of 100 different orientations to mimic the experimental conditions of a freely rotating bowtie in water.

FIGS. 48A-48L. Simulated g-factor spectra for a twisted left-handed sheet. Modeled geometry and the corresponding g-factor and extinction spectra for different FIGS. 48A-48C, pitch, FIGS. 48D-48F, thickness, FIGS. 48G-481, length, FIGS. 48J-48L, width. Decreasing pitch and thickness blue shifts the spectra, while increasing length and width red shifts the spectra. Modeled geometries are ideal shapes without strong scattering on edges. Hence the g-factors are an order of magnitude higher as observed in experiments.

FIGS. 49A-49E. Dipolar versus quadrupolar contribution to scattering cross-section. FIG. 49A, (top to bottom) modeled geometry of bowties from length 250 nm to 1000 nm. FIG. 49B, fraction of dipolar (electric+magnetic) scattering versus quadrupolar (electric+magnetic) scattering for a left-handed bowtie interacting with linearly polarized (LP) vs right-handed circularly polarized (RCP) versus left-landed circularly polarized (LCP) electromagnetic wave. Total scattering cross-section is decomposed into the constituent electrical (Cₚ), magnetic (Cₘ) dipole and electrical (C_{Oe}), magnetic (C_{Om}) quadrupole scattering cross-sections for FIG. 49C, linearly polarized, FIG. 49D, Right-circularly polarized (RCP) and FIG. 49E, Left-circularly polarized (LCP) electromagnetic wave.

FIGS. 50A-50E. Models for bowtie with variable size. FIG. 50A, Upon scaling the size of the bowtie from 250 nm to 1000 nm, the width, length, thickness and pitch increase simultaneously as shown here. FIG. 50B, the maximum g-factor spectra red-shifts from 400 nm to 800 nm to 1200 nm as the size increases from 250 nm to 1000 nm. FIG. 50C, Correspondingly the differential absorption peaks at the same wavelength as the size of the bowtie, while the differential scattering of the bowtie peaks at slightly smaller wavelengths as compared to absorption. FIG. 50D, Extinction spectra for the left-handed twisted sheet upon interaction with LCP and RCP. FIG. 50E, Contribution from absorption and scattering to extinction cross-section when LCP interacts with a left-handed twisted sheet.

FIGS. 51A-511. Scattered electromagnetic field around bowties. The field with the isoline at 2.5 (V/m)² is plotted around a left-handed twisted sheet model with a length of 1000 nm for FIGS. 51A, 51D, 51G RCP (blue) FIGS. 51B, 51E, 51F LCP (red) of wavelength FIGS. 51A, 51B, 385 nm, FIGS. 51D, 51E, 550 nm, FIGS. 51G, 51H, 1137 nm. The scattered field from LCP and RCP are overlaid in panels FIGS. 51C, 51F, 51I. At 385 nm differential scattered field is zero, at 550 nm it is maximum and at 1137 nm it is a local maximum. Note that the LCP scattered field (red) extends to farther regions in space than the RCP. At higher wavelengths the scattered field is dipolar in nature as seen from the single twisted lobe around the surface of the twisted sheet, while at lower wavelengths it is quadrupolar in nature as seen from two perpendicular lobes emanating from the top in panels FIGS. 51A-51F. Stronger coupling of LCP with LH sheet causes the scattering to be intense and continuous as compared to mismatch in handedness.

FIGS. 52A-52C. Polarization LIDAR setup for testing printed metasurfaces from bowties. Photographs of the optical arrangement for measuring the scattered rays from the bowtie coatings as seen from the FIG. 52A, top and FIG. 52B, side. FIG. 52C, Schematic of the polarization LIDAR setup with 1550 nm laser.

FIGS. 53A-53D. Bowties utilized in for printed metasurfaces tested by LIDAR with 1550 nm laser. SEM image of FIG. 53A, Cd₂(D-CST)₂ and FIG. 53B, Cd₂(L-CST)₂ bowties. FIG. 53C, CD spectra for the samples shown in panels FIGS. 53A and 53B. FIG. 53D, Coating of L-bowtie dispersed in PAA on cloth and the corresponding LIDAR backscattered signal of 1550 nm wavelength. Scale bars are 5 μm.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

In various aspects, the present disclosure provides chiral nanostructured microparticles having a bowtie shape with widely variable and controllable pitch, size, thickness, and length. By bowtie shape, it is meant that a three-dimensional polyhedral structure is formed having a shape that resembles a bowtie or hourglass, for example, having a pinched or restricted central region with connected conical shapes or flares at the terminal ends. In various aspects, a chiral microparticle may comprise an assembly of a plurality of nanoribbons. The nanoribbons are assembled together and define a bowtie shape that exhibits a predetermined chirality. The chirality may be controlled by adjusting pitch, size, thickness, and length of the microparticle assembly of nanoribbons. In certain aspects, each nanoribbon of the plurality of nanoribbons comprises at least two cystine molecules interconnected by at least one cadmium ion (Cd²⁺). Cystine (CST) is a dipeptide of cysteine amino acid bonded via an S—S bridge, which may be interconnected by Cd²⁺ ions. In other variations, the nanoribbons may comprise other peptides. In one alternative variation, the nanoribbon may comprise homocystine.

When light is directed at the chiral microparticles, a chirality exhibited may induce right-circular polarization, left-circular polarization, elliptical polarization, linear polarization (e.g., s or p type linear polarization), or any other suitable type of polarization known in the art. Similarly, when chiral microparticles prepared in accordance with the present disclosure are incorporated into a device, for example, as part of a polarizer component, they may generate right-circular polarized light, left-circular polarized light, elliptically-polarized light, linearly-polarized light (e.g., s or p type linearly polarized light), or any other type of polarized light known in the art. Such polarized light may be detected via a detector in the device. According to some examples, the polarization of a light beam (i.e., a combination of two or more light pulses) may be modulated from pulse to pulse, for example, to obtain additional information about one or more objects under consideration.

The self-limited assembly of such anisotropic building blocks (nanoribbons) makes possible high synthetic reproducibility, size monodispersity and computational predictability of their geometries for different assembly conditions. In certain aspects, they display multiple strong circular dichroism peaks originating from absorptive and scattering phenomena. Unlike classical chiral molecules, these particles display a continuum of Osipov-Pickup-Dunmur (OPD) chirality measures that exponentially correlate with the spectral positions of the circular dichroism peaks. In certain aspects, the bowtie microparticles may exhibit a chirality in terms of OPD index of −150 to +150, optionally −100 to +100, optionally −75 to +75, optionally −50 to +50, optionally −25 to +25, optionally −15 to +15, optionally −10 to +10. In terms of enantiomeric excess, chirality may range from −1 to +1. Bowtie particles with variable polarization rotation may be utilized in printing photonically active metasurfaces with spectrally tunable positive/negative polarization signatures for light detection and ranging (LIDAR) devices.

In certain aspects, the assembly of the bowtie microparticles can proceed by the following process. Bowties microparticles are assembled from (stacked) nanoribbons. The nanoribbons are assembled from nanoplatelets. Nanoplatelets that may have an average thickness of greater than or equal to about 1 nm to less than or equal to about 2 nanometers are observed from nanoclusters, where the nanoclusters have helical molecular motifs in them. In certain variations, the microparticle assembly having a bowtie shape may have greater than 1 nanoribbon to less than or equal to about 10,000 nanoribbons.

Each respective nanoribbon may have a length of greater than or equal to about 10 nm to less than or equal to about 100 micrometers, optionally greater than or equal to about 10 nm to less than or equal to about 50 micrometers, optionally greater than or equal to about 10 nm to less than or equal to about 25 micrometers, optionally greater than or equal to about 10 nm to less than or equal to about 10 micrometers, optionally greater than or equal to about 10 nm to less than or equal to about 5 micrometers, and in certain aspects, optionally greater than or equal to about 10 nm to less than or equal to about 2 micrometers.

Each respective nanoribbon may have a thickness of greater than or equal to about 50 nm to less than or equal to about 10 micrometers, optionally greater than or equal to about 100 nm to less than or equal to about 10 micrometers, optionally greater than or equal to about 250 nm to less than or equal to about 10 micrometers, and in certain aspects, optionally greater than or equal to about 500 nm to less than or equal to about 10 micrometers.

Each respective nanoribbon may have a width of greater than or equal to about 3 nm to less than or equal to about 20 micrometers, optionally greater than or equal to about 3 nm to less than or equal to about 15 micrometers, optionally greater than or equal to about 5 nm to less than or equal to about 10 micrometers, optionally greater than or equal to about 5 nm to less than or equal to about 5 micrometers, optionally greater than or equal to about 5 nm to less than or equal to about 3 micrometers, and in certain aspects, optionally greater than or equal to about 10 nm to less than or equal to about 1 micrometer.

After the nanoribbons are assembled into the bowtie shaped microparticle, the microparticle may have a length of greater than or equal to about 100 nm to less than or equal to about 100 micrometers, optionally greater than or equal to about 100 nm to less than or equal to about 75 micrometers, and in certain aspects, optionally greater than or equal to about 100 nm to less than or equal to about 50 micrometers.

The bowtie shaped microparticle may have a thickness of greater than or equal to about 50 nm to less than or equal to about 10 micrometers, optionally greater than or equal to about 100 nm to less than or equal to about 10 micrometers, optionally greater than or equal to about 250 nm to less than or equal to about 10 micrometers, and in certain aspects, optionally greater than or equal to about 500 nm to less than or equal to about 10 micrometers, optionally greater than or equal to about 10 nm to less than or equal to about 5 micrometers, and in certain aspects, optionally greater than or equal to about 10 nm to less than or equal to about 2 micrometers.

The bowtie shaped microparticle may have a width of greater than or equal to about 50 nm to less than or equal to about 30 micrometers.

The bowtie shaped microparticle may have a pitch of greater than or equal to about 10 nm to less than or equal to about 100 micrometers, optionally greater than or equal to about 10 nm to less than or equal to about 50 micrometers, optionally greater than or equal to about 10 nm to less than or equal to about 5 micrometers or alternatively, optionally greater than or equal to about 200 nm to less than or equal to about 100 micrometers.

In certain aspects, methods of making the microparticles having a bowtie shape include coordinating cystine with cadmium chloride solution in the presence of water and sodium hydroxide (NaOH). Water can be replaced or supplemented with other solvents, such as ethanol, methanol, dimethyl formamide, dimethyl sulfoxide, acetonitrile, and the like. Cadmium metal serves as the bridge between cystine molecules (or peptides). In certain alternative variations, as discussed above, cystine can be replaced with longer chain homocysteine, by way of non-limiting example.

In other variations, the present disclosure contemplates a chiral dispersion comprising a plurality of chiral microparticles each comprising a plurality of nanoribbons distributed in a medium. In certain aspects, distinct chiral microparticles may be included in the dispersion to tailor optical properties. For example, the plurality of chiral microparticles may include at least one first chiral microparticle in the plurality of chiral microparticles exhibits a first chirality property that is distinct from a second chirality property exhibited by at least one second chiral microparticle in the plurality of chiral microparticles. For example, the at least one first chiral microparticle may have a first size, first twist angle, first pitch, or other property, while the at least one second chiral microparticle may have a distinct second size, second twist angle, second pitch, and the like.

As further background, mathematical definitions of mirror asymmetry recognize the continuity of chiral geometries that can be visualized by gradual stretching of macroscale helical springs to obtain different periodicity of coils, i.e. pitch. At smaller scales, continuously variable chirality can be observed for origami/kirigami sheets, nanocomposites and some polymeric solids whose shape and circular dichroism can be varied by mechanical deformation and external fields. However, chirality in chemistry commonly manifests as a binary property. Small chiral molecules are typically known as D/L or R/S enantiomer, while larger molecules with helical shapes are often denoted as A/A or M/P enantiomers. The binary chirality of, for instance amino acids, originates from high-energy penalty for distorting the optical center based on sp³ carbon atoms. The discreteness of chirality in liquid crystals, macromolecular compounds, helical polymers and nanoparticles (NPs) manifests in abrupt transitions between chiral phases with different crystallinity or particle shapes. Energy penalties are relaxed for large flexible molecules, supramolecular complexes, and biopolymers, but the restrictions on chiral shapes remain nevertheless stringent. Unlike macroscale springs, the helical pitch across the entire variety of biomolecules vary little. For DNA, α-helixes, and β-sheets it changes only in the range of 11-46 Å, 2.3-5.5 Å, and 7-8 Å respectively, which is essential for precise folding of biomolecules.

The transition from discrete chiral phases and shapes to a palette of chemical compounds with widely and continuously dialed chirality is important for the development of rapidly evolving areas of chiral photonics and chiral metamaterials, as well as for established fields of biochemical separations and chiral catalysis. From a fundamental perspective, availability of such compounds is also a prerequisite for establishing correlations between chirality measures and chemical properties. Even for optical activity, the attempts to correlate it with a variety of chirality measures largely failed, so far.

In the context of the present disclosure, continuously variable chiral geometries are possible for nanostructured microparticles with bowtie shapes. As discussed above, these microparticles represent hierarchically assembled nanoribbons containing helical chains of cystine (CST, the dipeptide of cysteine bonded via S—S bridge) interconnected by Cd²⁺ ions. The finely controlled balance between short- and long-range interactions and defect tolerance of their assembly process enable preparation of bowties with widely tunable twist angle, pitch, size, thickness, and length.

### Synthesis and Structural Characterization.

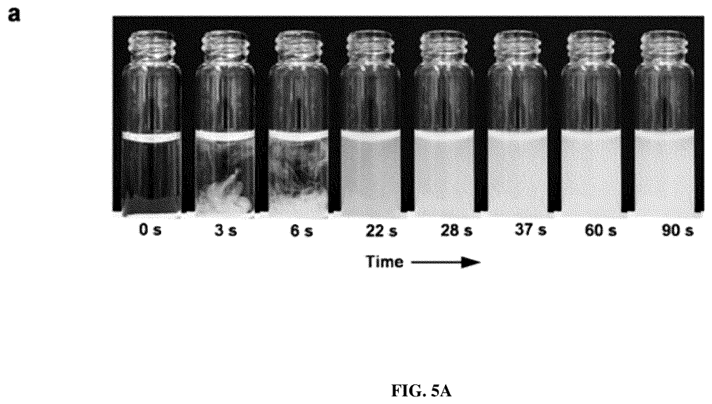

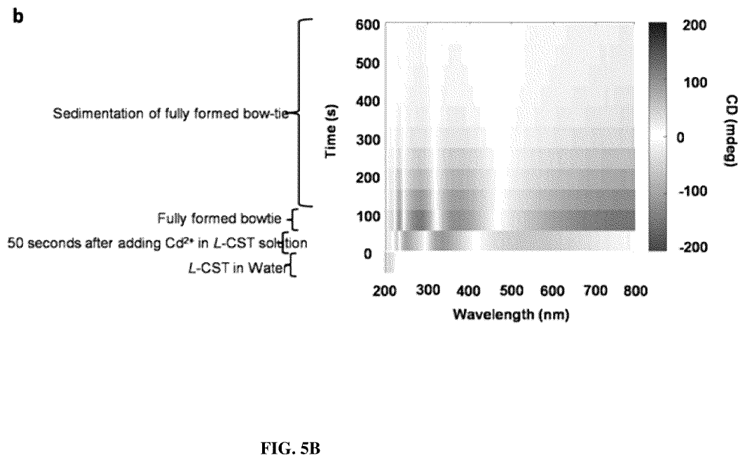

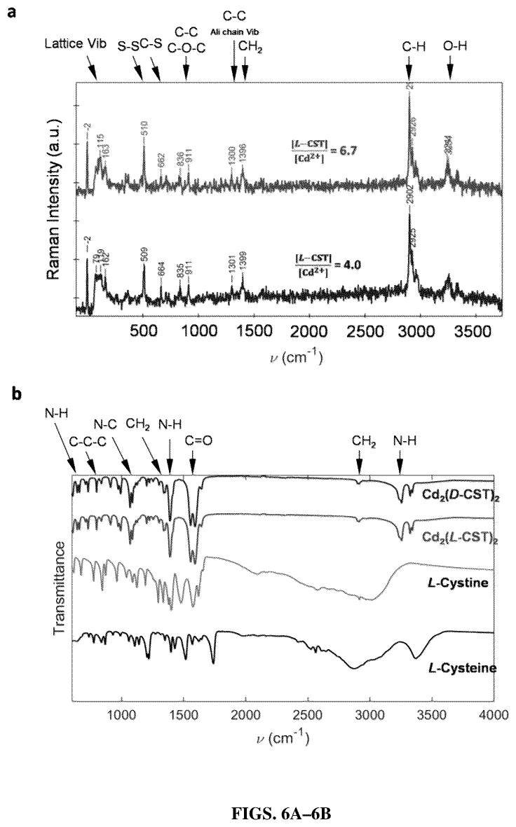

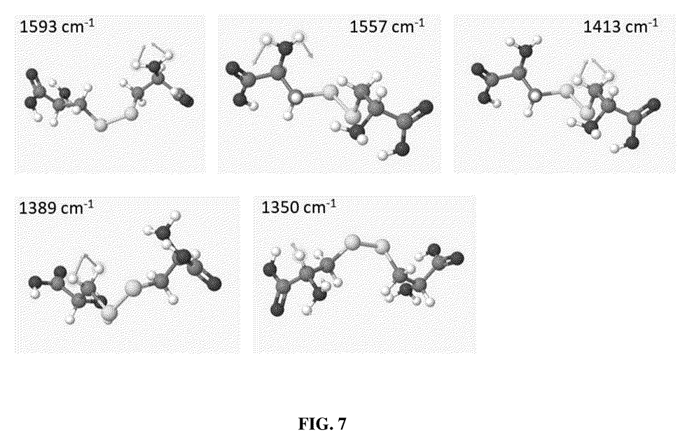

Bowties are synthesized in one variation by mixing an aqueous solution of Cd²⁺ with aqueous solution of L- or D-cystine (CST) (FIGS. 5A, 5B). Cadmium chloride (CdCl₂), 99.99% trace metals basis, L-CST, ≥98% (TLC), crystalline, D-CST, 98%, sodium hydroxide (NaOH) pellets >97% were purchased from Sigma-Aldrich. De-ionized water (18.2-mΩ-cm) was used for the preparation of stock solutions and aqueous dispersions. 10 mL stock solutions of CdCl₂ (0.1 M), L-CST (0.1 M), D-CST (0.1 M) and NaOH (2.5 M) were prepared by dissolving the required amounts in DI water. pH of CST stock solution was raised to 11 by adding 1 mL of 2.5 NaOH in 10 ml of solution. An immediate change in appearance was observed by the formation of a clear solution. Remaining solids were dissolved by mild-sonication for 10 seconds. Bowties were synthesized by the mixing stock solutions of CdCl₂ and L-CST or D-CST in 1:1 stoichiometric ratio in an aqueous media. Typical synthesis involved 20 μL of L-CST added to 960 μL of water followed by the addition of 20 μL CdCl₂. The solution mixture was shaken vigorously until the dispersion turned milky, which is an indicator of the bowties assembly process. After that, the mixture was kept still at room temperature for 15 minutes to ensure the completion of the self-assembly. The dispersion was subsequently centrifuged three times in DI water at 6000 rpm for 3 mins. Final aqueous dispersion was stored at room temperature and used for further characterization and studies.

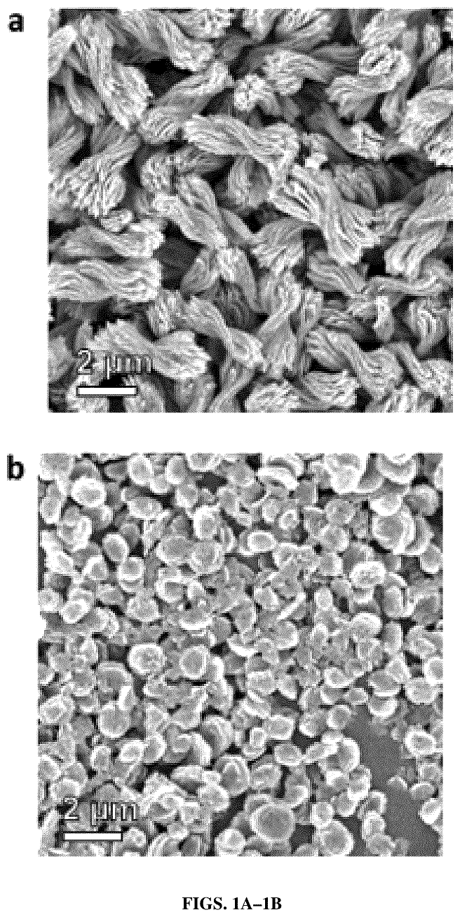

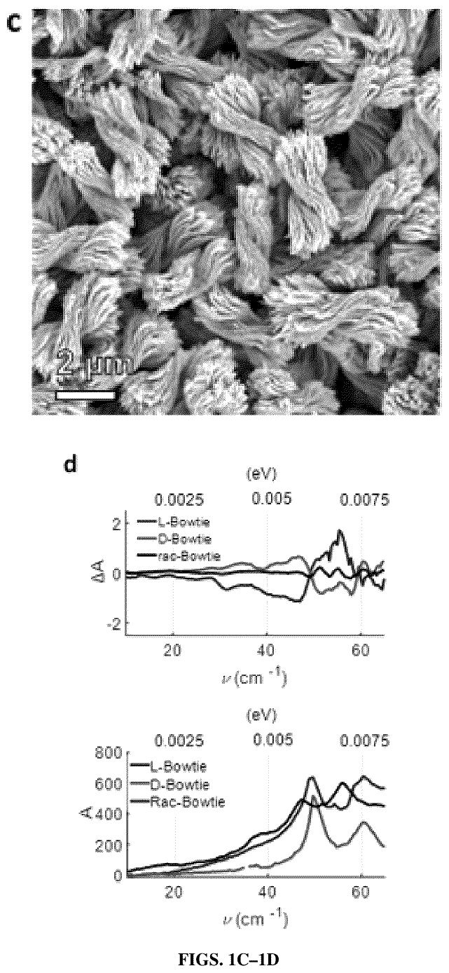

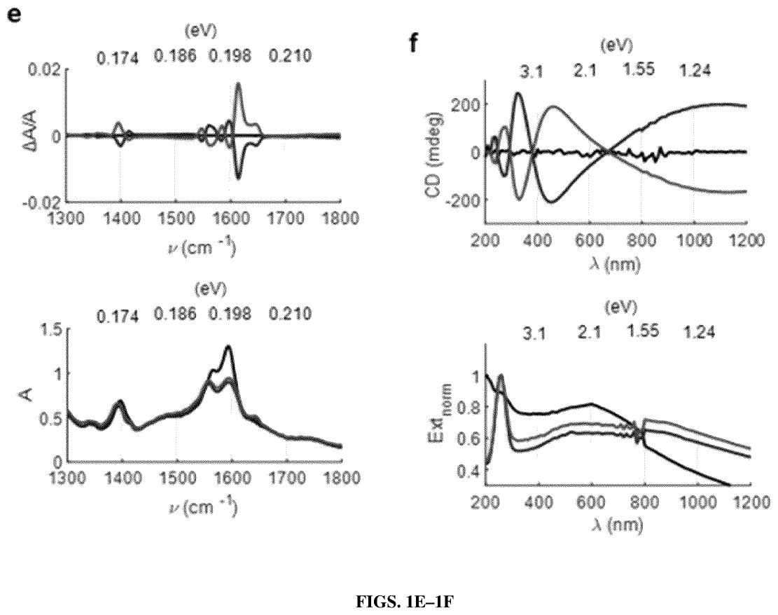

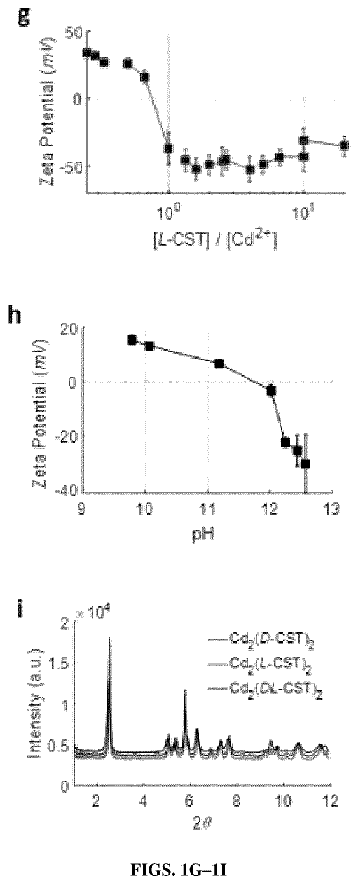

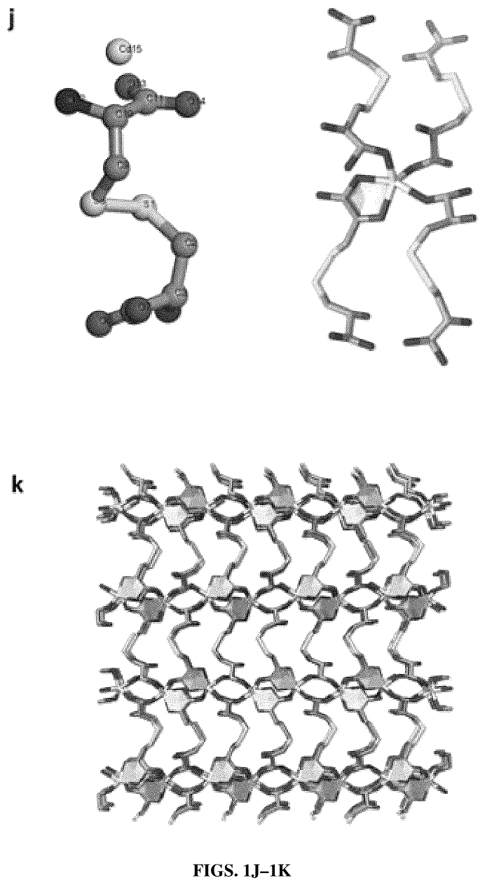

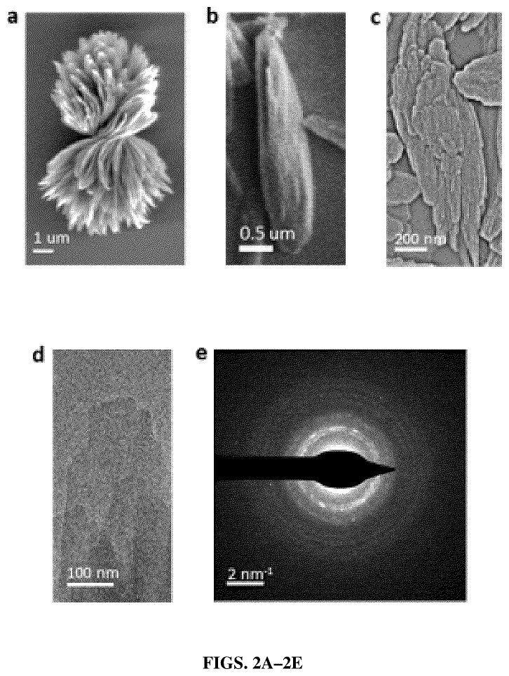

For example, 10 μl of (1 mM) CdCl₂ and 10 μl of (1 mM) L-CST were added to 1 ml of water at room temperature and pH 11, which resulted in bowties with pitch (p), width (w), thickness (t), and length (l) of 4.1 μm, 1.3 μm, 0.5 μm, 3.1 μm, respectively (FIG. 1A). The synthesis of the bowtie particles is 100% enantioselective with L- and D-CST resulting in correspondingly left- and right-twisted bowties exclusively (FIGS. 1A, 1C). The resulting particles are uniform in shape, size and handedness with standard deviation in p, w, t, and l equal to 19.0%, 13.6%, 11.9%, and 11.5%, respectively. Relative monodispersity of the bowtie particles indicates that they are formed in a self-limited assembly process (FIGS. 16A-16B) which makes them suitable for scalable production.^{5,6} Nanostructured microparticles resembling a stack of flat nanoscale ‘pancakes’ are observed when rac-CST is used (FIG. 1B).

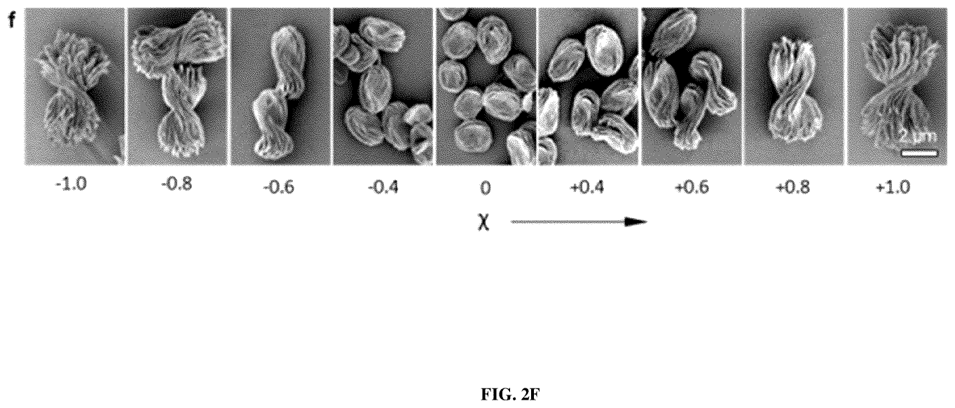

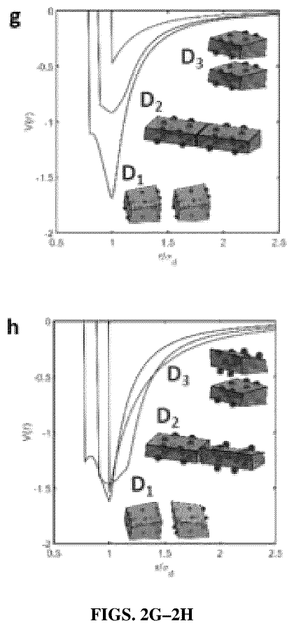

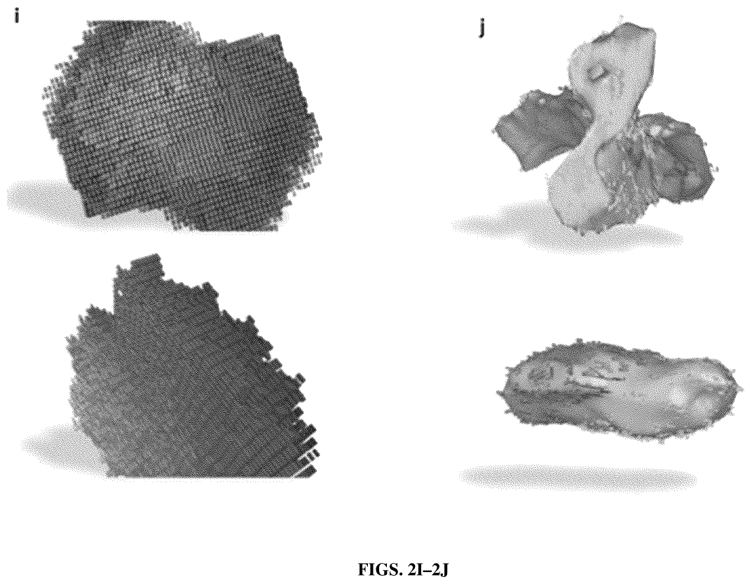

Electron microscopy, X-ray diffraction (XRD), and electron diffraction identify several levels of hierarchical organization in the cadmium cystinate bowties (FIG. 1I, FIG. 2A). Scanning electron microscopy (SEM) images for the terminal (FIGS. 1A-1C) and intermediate (FIGS. 2A, 2B) stages of the synthesis show that the bowtie particles are structured as stack of twisted nanoribbons with 200-1200 nm in length and 45 nm in thickness. In turn, the nanoribbons are assembled from nanoplatelets with 50-200 nm in length (FIGS. 2C, 2D) and a thickness of ˜1.2 nm. The nanoribbons and bowties acquire progressively stronger twist as the enantiomeric excess (±χ) of L- or D-CST increases (FIG. 2F.).

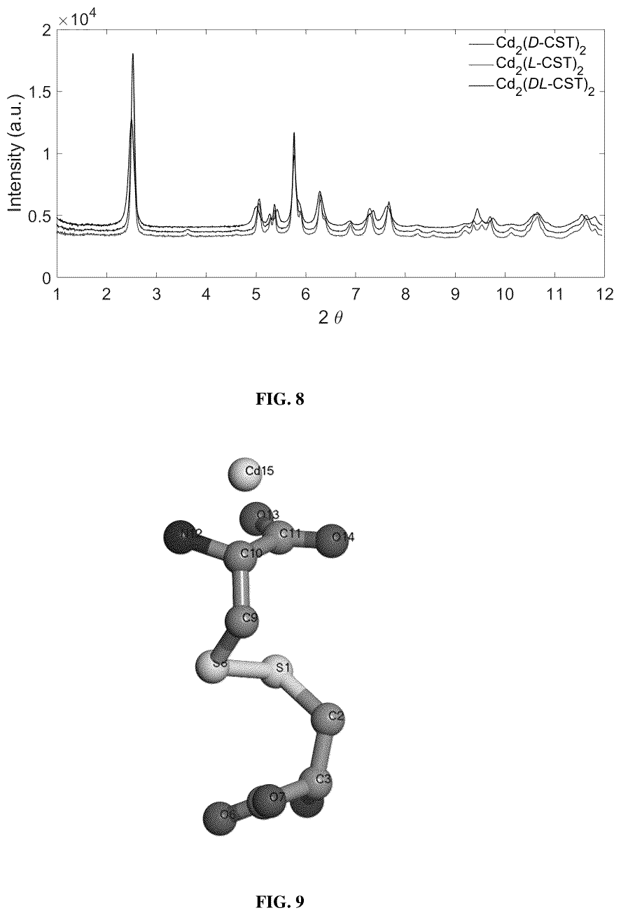

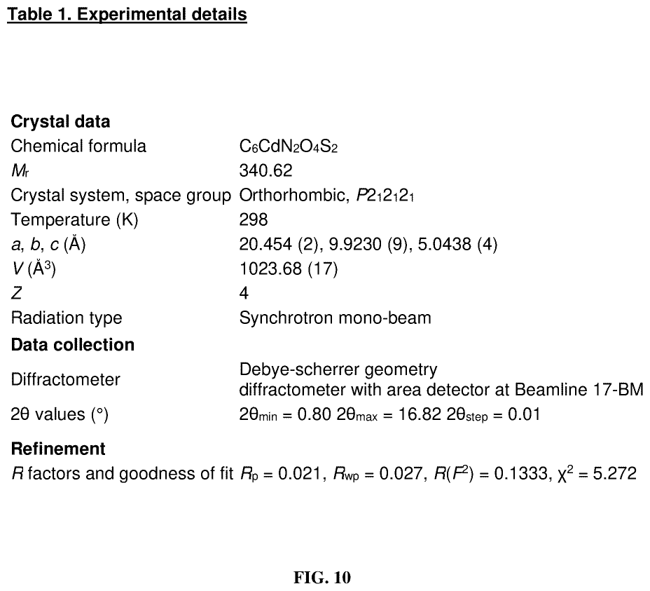

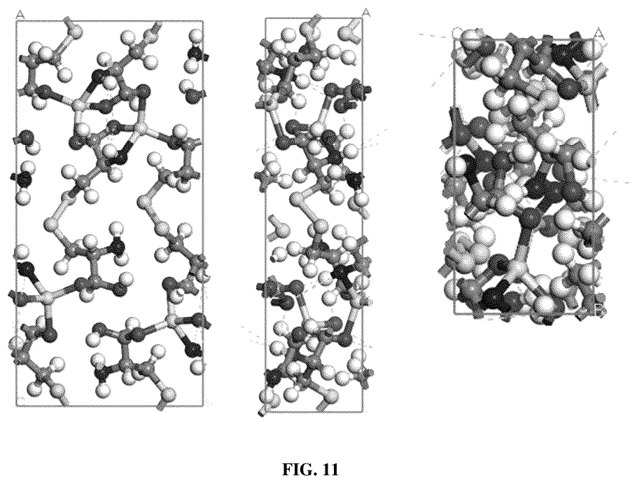

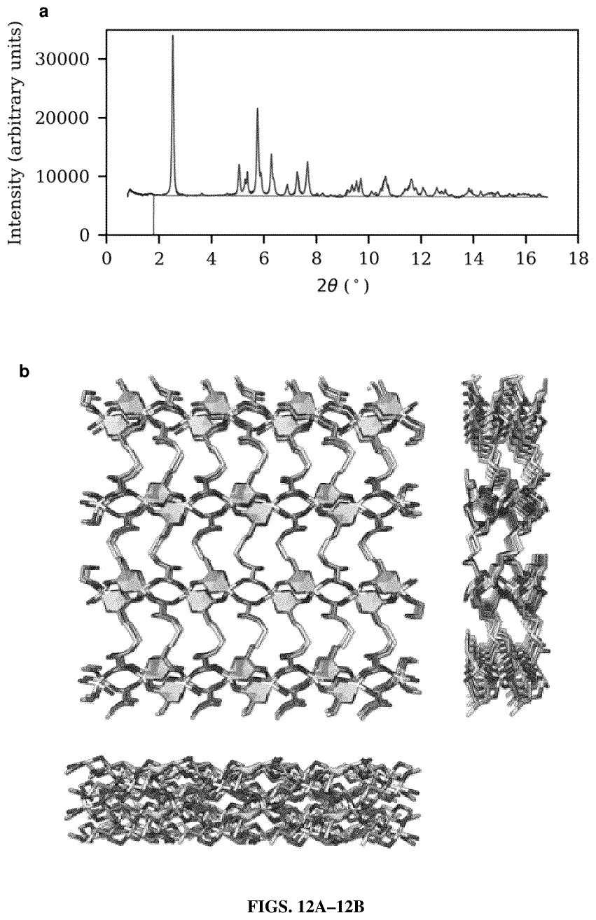

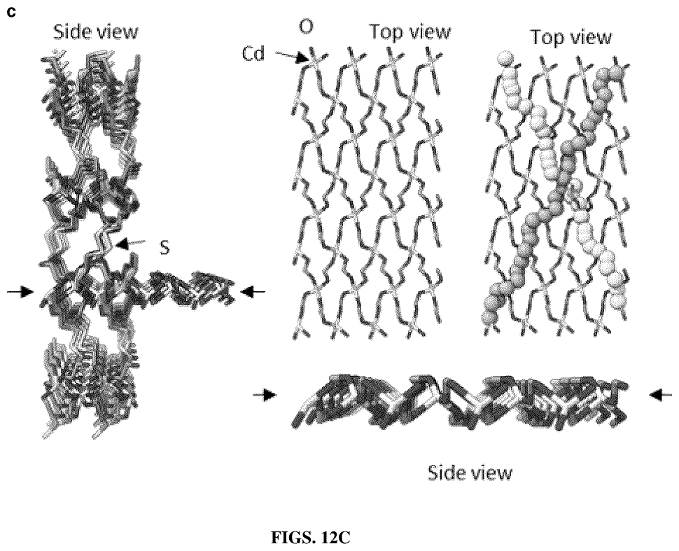

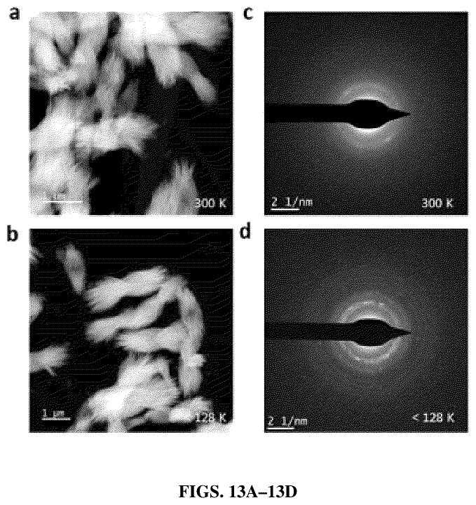

To understand better how atomic structure of the bowtie particles can accommodate their continuous chirality while remaining chemically stable, synchrotron XRD patterns for particles in FIGS. 1A-1C are obtained. Structure solution followed by Reitveld refinement suggested several polymorphs of L-CST coordinated with Cd atoms (FIGS. 8-12, including Table 1 in FIG. 10), indicating a possibility of variable atomic organization. The cumulative analysis of the XRD models points to helical chains from Cd₂CST₂ units as the elementary structural unit of the nanoribbons (FIGS. 12A-12B). These ‘building blocks’ (FIGS. 1J, 1K, FIGS. 12A-12B) allows for partial swapping of L-CST for D-CST without drastic disruption of the short-range coordination bonds. The amine and carboxylic groups located on the exterior of the helical chains can form hydrogen bonds with neighboring ones stabilizing the structure and facilitating the nanosheet stacking (FIGS. 1J, 1K, 2C-2D). XRD data indicate that atomic organization in nanosheets is imperfect as can be evidenced by peak overlap below 2.5 Å, but this feature is needed for the chemical structure to produce the multiplicity of twisted shapes.

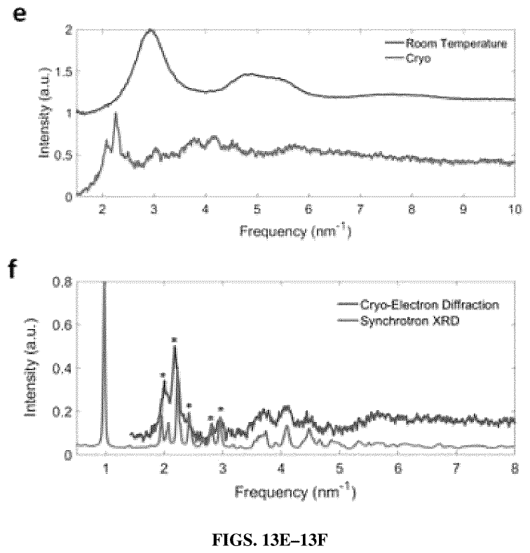

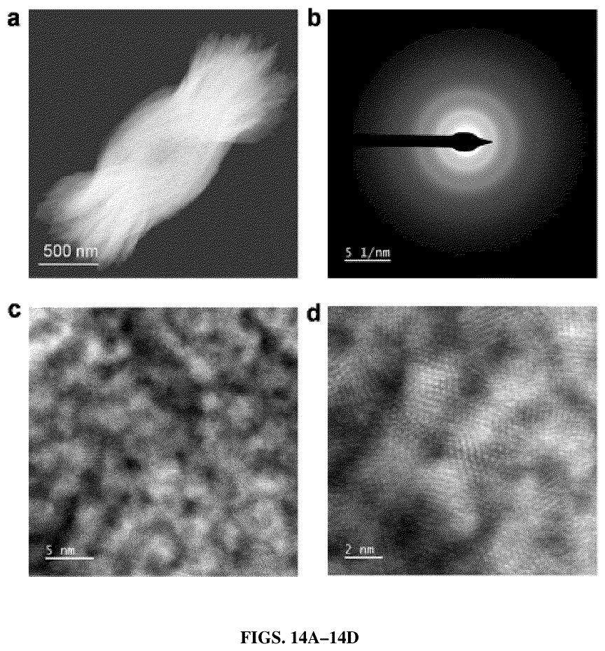

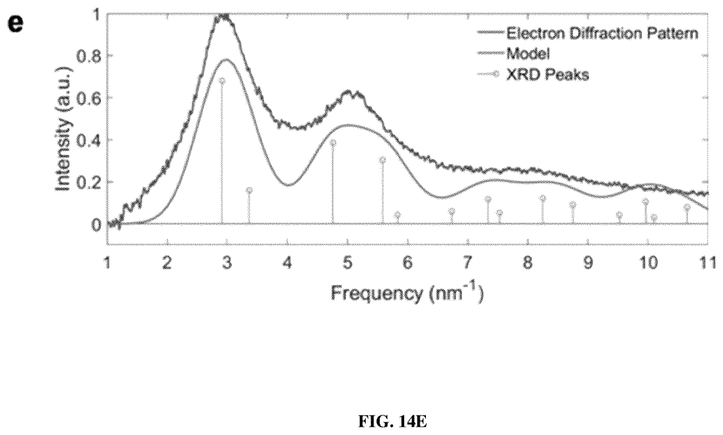

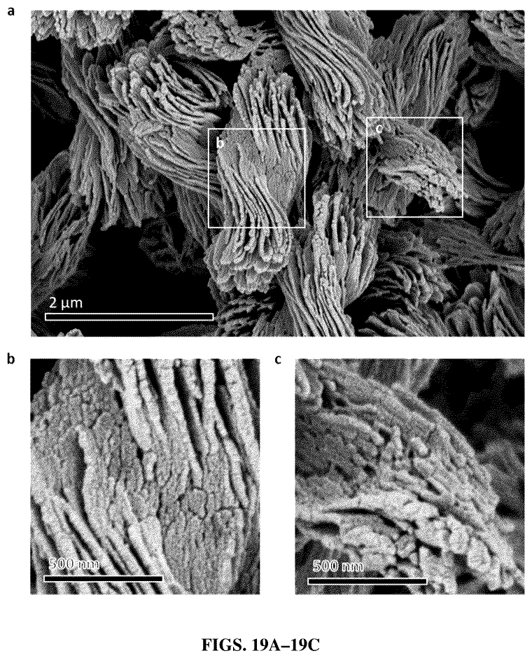

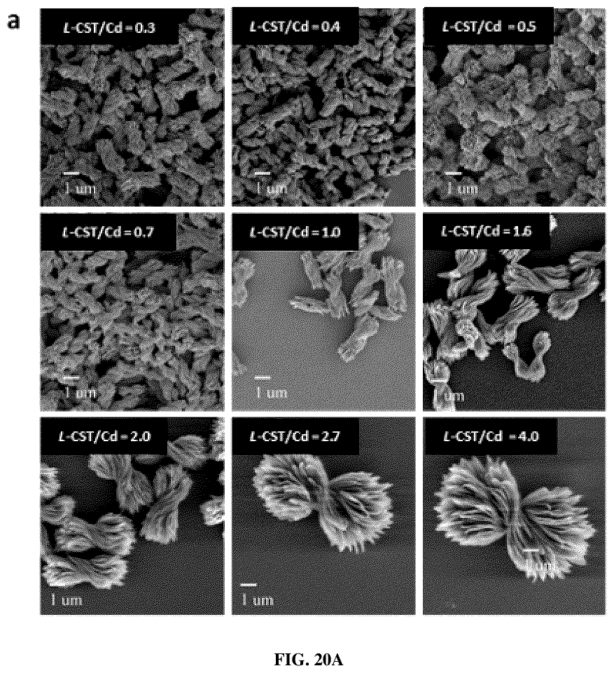

Selected area electron diffraction (SAED) from a single nanoribbon confirms the structural analysis based on XRD (FIG. 2E). Cryo-SAED patterns show characteristic reflexes at 4.9 Å, 4.6 Å, 4.1 Å, which is consistent with distances between planes in the orthorhombic Cd₂CST₂ structure at 5.1 Å, 4.5 Å, 4.1 Å (FIGS. 13A-13D). The nanoribbons are polycrystalline revealing fairly ordered domains of helical chains as observed from sharp spots in the SAED pattern (FIG. 2E, FIGS. 19A-19C). Some degree of polycrystallinity affords the nanosheets to accommodate CST ligands of opposite chirality, while retaining the physical integrity of the particle.

The flexible hydrogen bonded networks between the nanosheets can accommodate variable bond angles and is one of the factors enabling variable chirality. The ability of constituent nanoplatelets and nanoribbons of cadmium cystenate to ionize—as can be evidenced by the high zeta-potential (FIG. 1G, 1H) is also significant. The surface potential of nanoscale particles and resulting long-range repulsive interactions can be varied in a wide range by pH and ionic strength enabling variability of the bowtie geometries. The twist of the nanoribbons make the electrostatic interactions chiral that reinforces the handedness of their stacking and therefore the similarity of the particle shapes. Other contributing interactions include mechanical deformations of the nanoribbons that accommodate the atomic and nanoscale preferences of the two-dimensional assemblies.

Chiroptical activity. Extinction and circular dichroism (CD) spectroscopies from ultraviolet to terahertz (THz) frequencies demonstrate that the left- and right-handed bowties display a rich set of mirror symmetric bands, while the ‘pancake stack’ assemblies are chiroptically silent (FIGS. 1D, 1F). Visible, Raman, FTIR, mid-IR and THz spectra (FIGS. 1D-1F) confirm the molecular structure of the bowtie particles established by XRD and SAED. Raman spectra show characteristic peaks at 510 cm⁻¹ corresponding to S—S disulfide stretches. The strong and wide band from 3000 to 3750 cm⁻¹ observed in FTIR spectra confirm the multiplicity of hydrogen bonds involved in stabilization of the nanosheets and their stacks. The UV CD spectra have multiple positive and negative peaks denoted as P1 (−, 1040 nm), P2 (+, 460 nm), P3 (−, 330 nm), P4 (+, 270 nm), and P5 (−, 235 nm), from the longest to the shortest (FIG. 1F). Among them P5 and P4 in the UV part of the spectrum are attributed to electronic transitions in the CST ligands. They are broadened and red-shifted compared to free CST due to coordination with Cd²⁺ and exchange interactions with other states. VCD spectra are ca 100 times stronger compared to those of free CST due to long-range resonant coupling between helical chains forming cadmium cystenate nanosheets. The VCD band near 1600 cm⁻¹ is typically a simple bisignate peak for free CST, but it is split into five bands in the bowtie particles due to the asymmetric environment around the nitrogen and oxygen atom involved in coordination bonding with cadmium atom when forming the nanoribbons (FIGS. 1J, 1K, 9). THz circular dichroism shows the existence of chiral phonons propagating along the nanoribbons and nanosheets (FIG. 1D). Besides substantiating the hierarchical organization of the bowties, these data firmly indicate that the bowties made with L- or D-CST are true multiscale enantiomers for which the mirror asymmetry is present at all molecular, sub-nanometer, nanometer and micrometer scales.

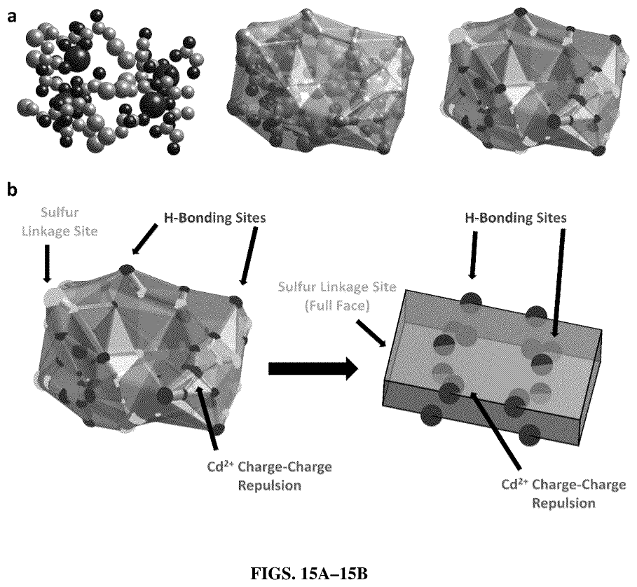

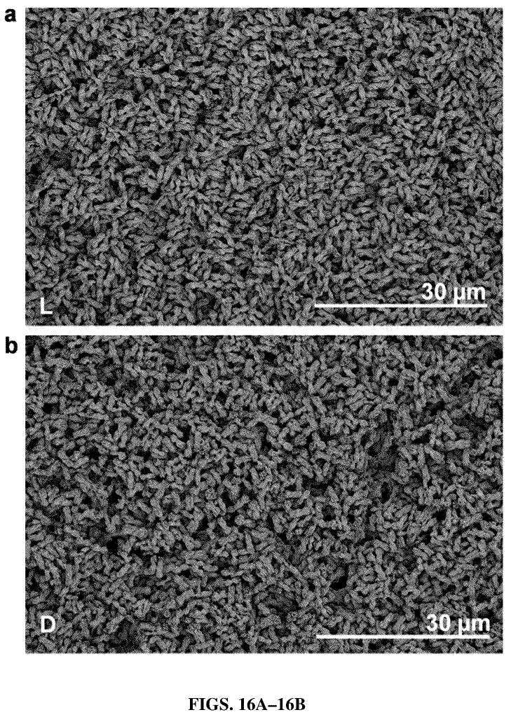

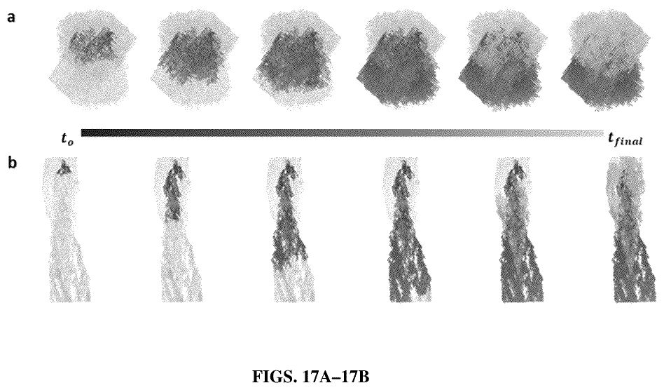

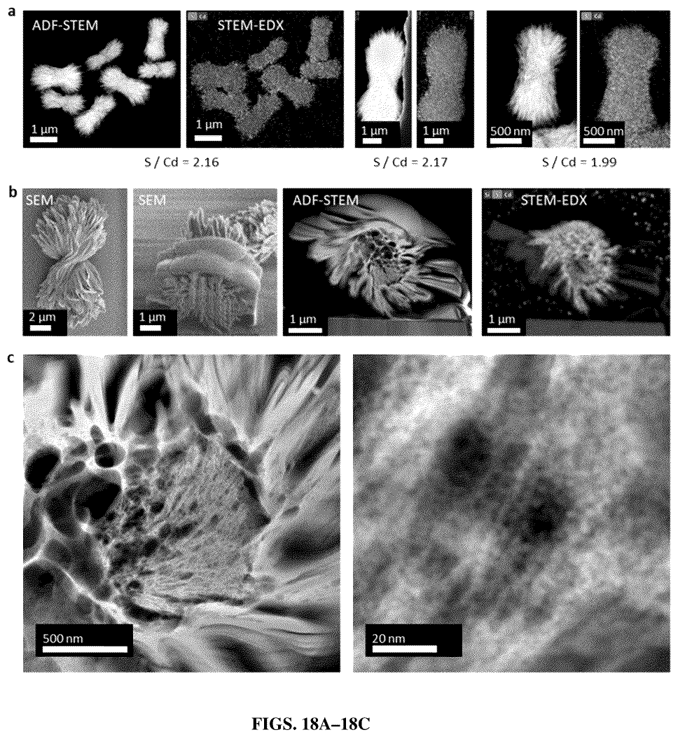

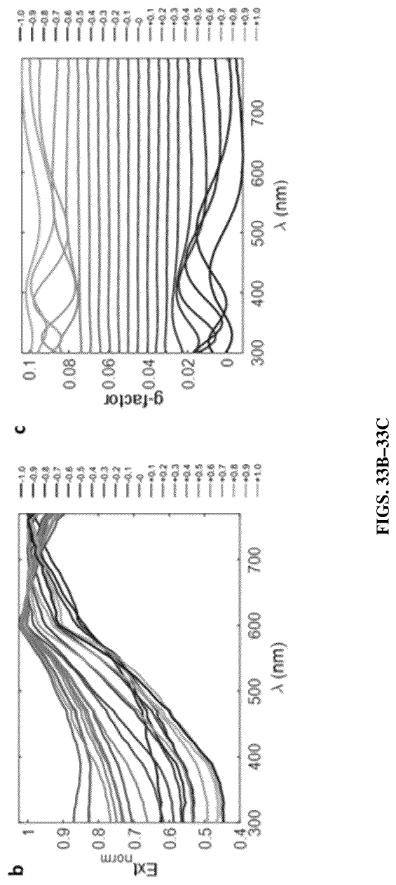

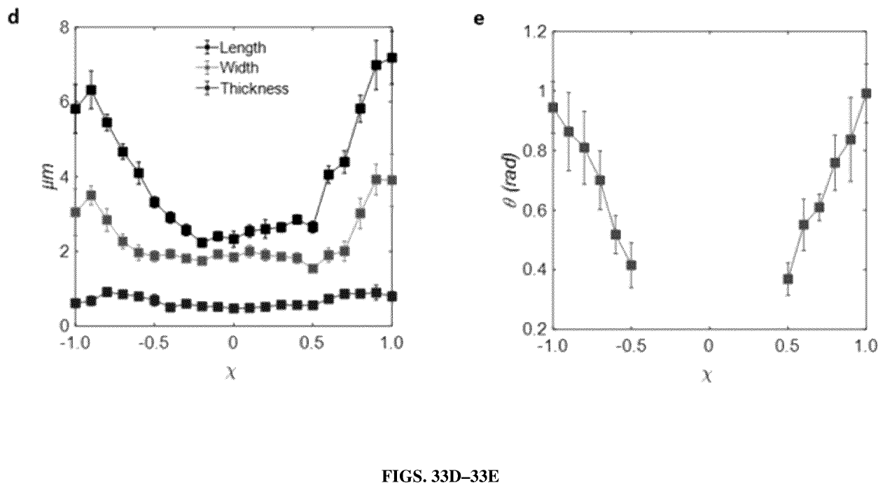

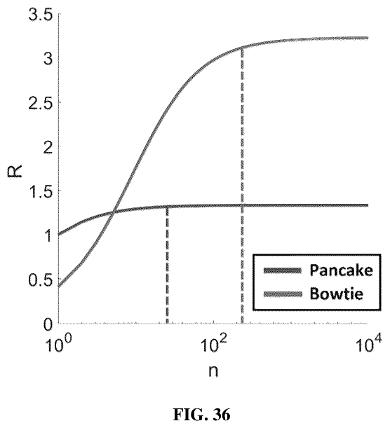

Modeling of bowtie self-limited assembly. Computational models of bowtie formation reveal the process of hierarchical self-assembly essential for their size/shape uniformity, high yield, chirality variability and transfer. Based on XRD, TEM, and spectroscopic data, a coarse-grained version of the nanocluster with helical chains (FIGS. 15A-15B) is constructed as the building block for nanoribbons and nanosheets (see Methods). A Monte Carlo (MC) growth algorithm was then employed to simulate bowtie assembly. Based on zeta potential measurements (FIGS. 1G, 1H, FIGS. 21, 22A-22B), an average net charge on each building block of the nanosheets is found to be +1. Electrostatically restricted self-assembly process implies that the addition of each nanocluster increases the total charge of the growing particle. Computation of the pair-wise potentials (FIGS. 2G, 2H) between clusters for L-monochiral and racemic versions of the nanoclusters, abbreviated as LCL or LCD, respectively, revealed growth directions along which they interact with each other through hydrogen bonding between —COOH and —NH₂ groups. These growth directions reflect the heirarchy of structural formation observed in experiments: that is, chains assembling into nanosheets that then stack to ultimately form the observed bowtie structures. As the number of nanoclusters and resulting nanoplatelets in the assembly increase, the size of the ordered domains in the nanosheets also increases (shown by color in FIG. 2I, FIGS. 17A-17B). The growth of nanoribbons stops when the net charge repulsion on the particle becomes strong enough to prevent further attachment of the nanocluster (FIG. 36), balancing out short-range coordination bonds, hydrogen bonding and vdW attraction. LCL nanoclusters form a twisted platelet (FIG. 2J, top row), while LCD ones form a flat sheet (FIG. 2J, bottom row). Quite remarkably, the model captures very well the fact that the enantiomeric composition of the nanoclusters, χ, determines the chirality of the bowtie microparticles propagating up in scale as the particles evolve in size. The high efficacy of such multiscale chirality transfer for different χ results in formation of bowties uniform pitch and size (FIG. 2F, FIGS. 33A-33E).

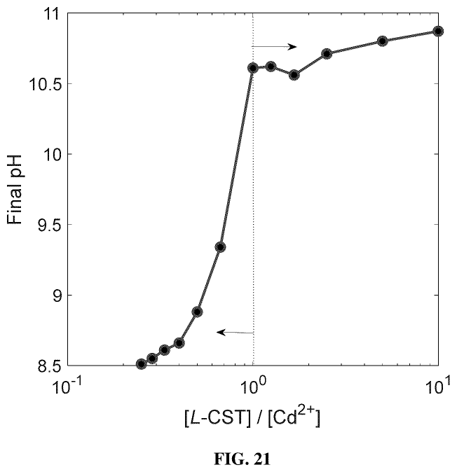

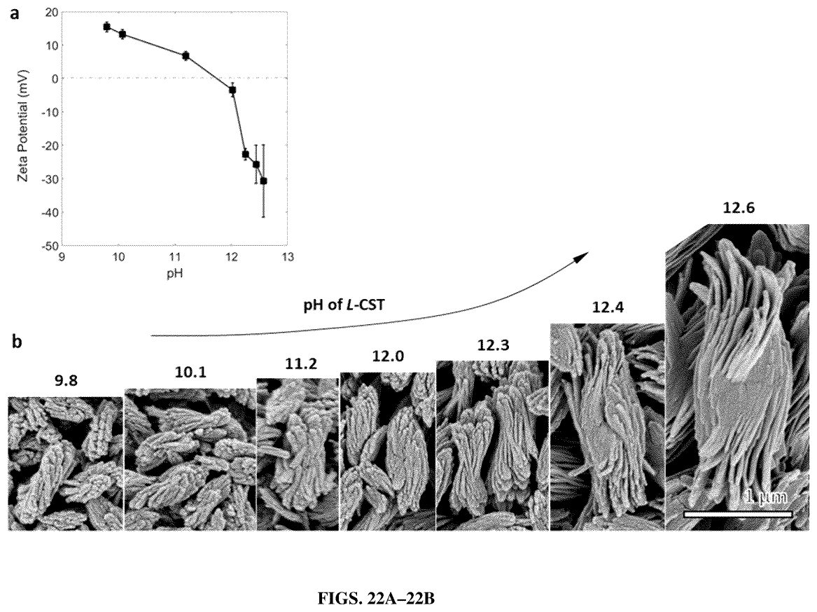

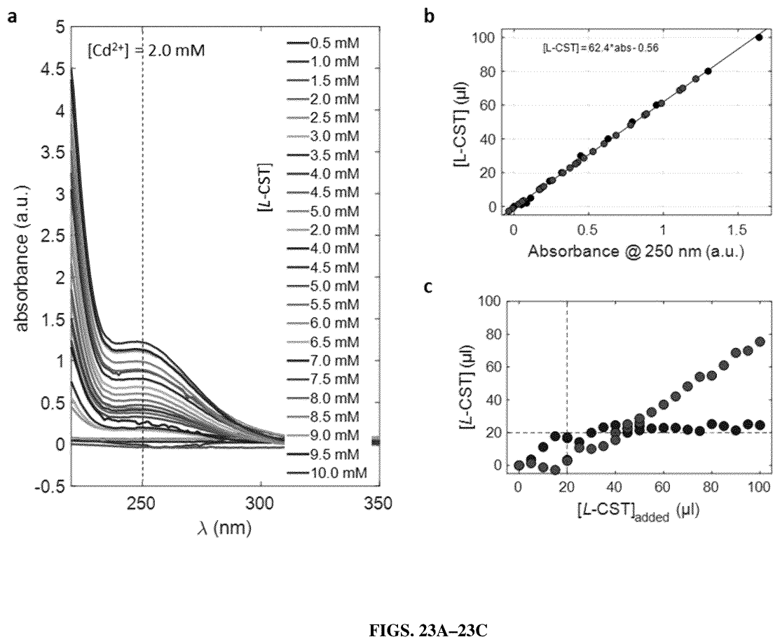

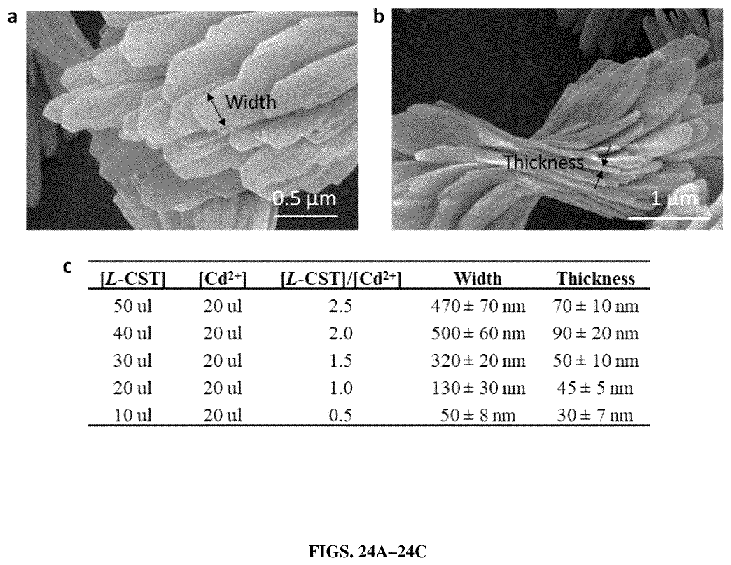

Chirality Continuum. Hierarchical assembly of electrostatically-restricted assemblies from helical nanoclusters, enables wide-range tuning of electrostatic repulsion, van der Waals, hydrogen bonding and other interactions, when changing temperature, solvent, salt concentration, chemical nature of the counter-ions, etc. Among the multiplicity of permutations of assembly parameters, it is instructive, to consider three prominent cases that can exemplify the effect of different interparticle interactions determining the bowtie geometry. (1) The increase of ionic strength results in the increase of l, w, and t because the range of electrostatic interactions is reduced when ionic strength and solvent polarity are increased (FIGS. 21, 22A-22B). (2) The increase of the zeta-potential with pH results in the increase of l, w, and t.

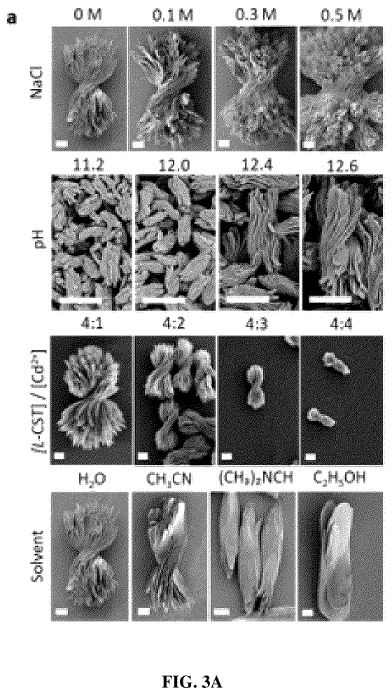

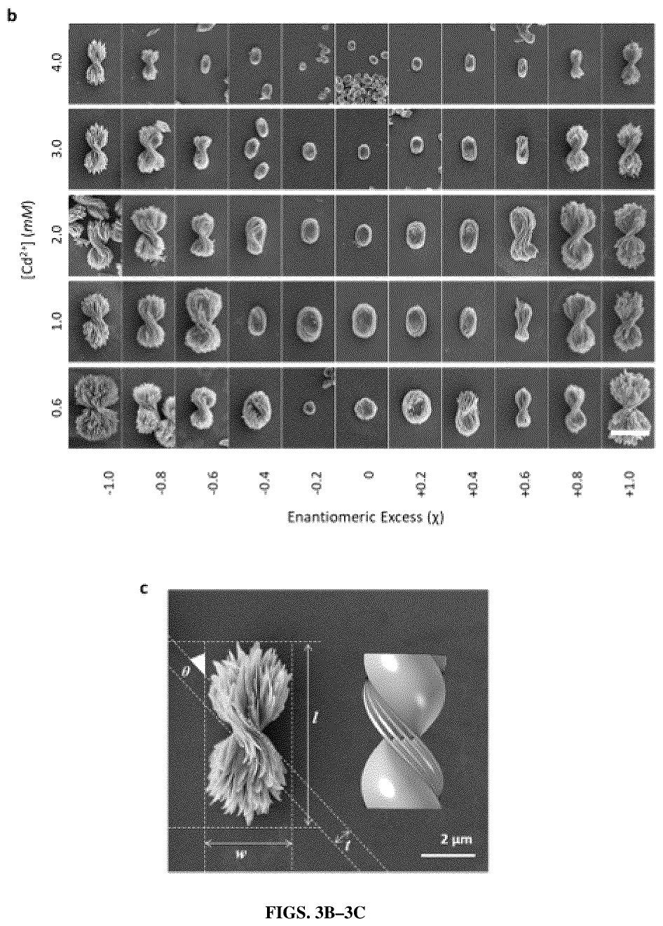

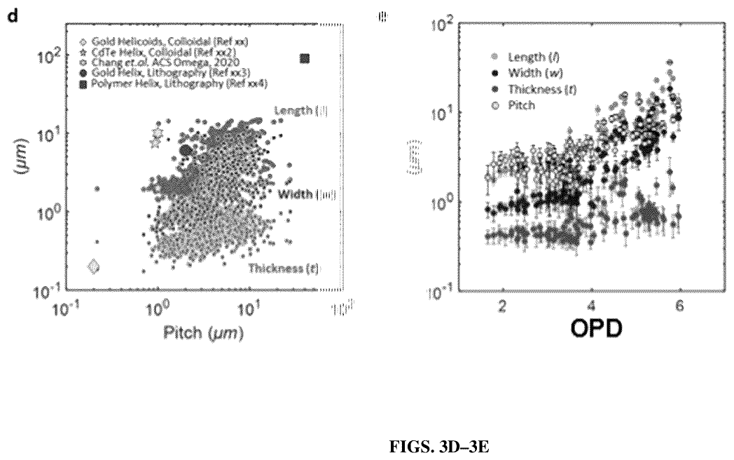

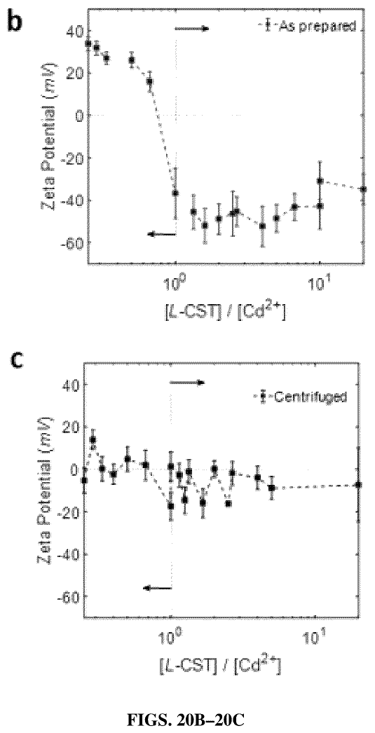

De-protonation of the —COOH groups on the surface of nanoribbons and nanoplatelets results in the concomitant reduction of the hydrogen bonding between the ribbons in the stack and, thus, in the greater separation between the nanosheets. (3) The increase of L-CST/Cd ratio leads to the increase of l, w, and t due to adsorption of the counter-ions on the bowtie surface (FIG. 18A). The increase of the CST concentration simultaneously promotes (FIG. 3A, FIGS. 20A-20C, 26) the close-range hydrogen bonding between the helical nanoclusters resulting in thicker and wider nanoribbons (FIG. 3A; FIGS. 24A-24C).

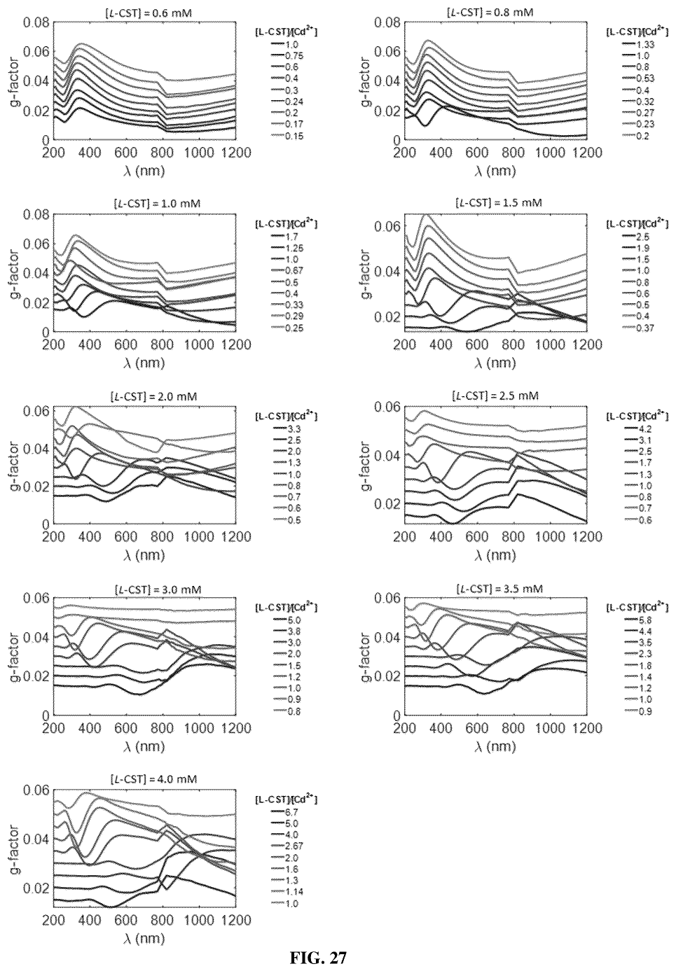

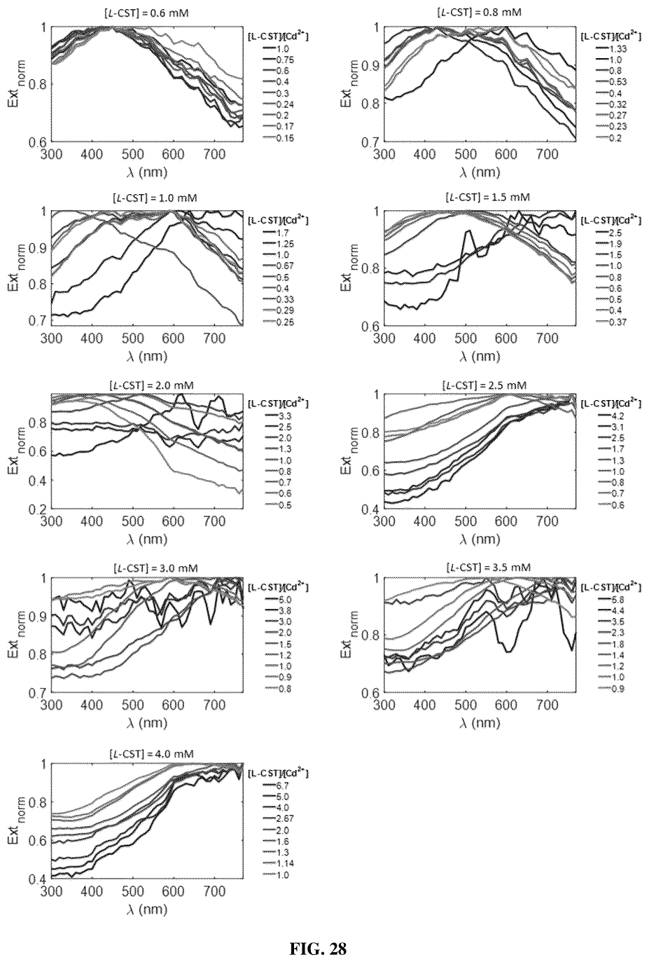

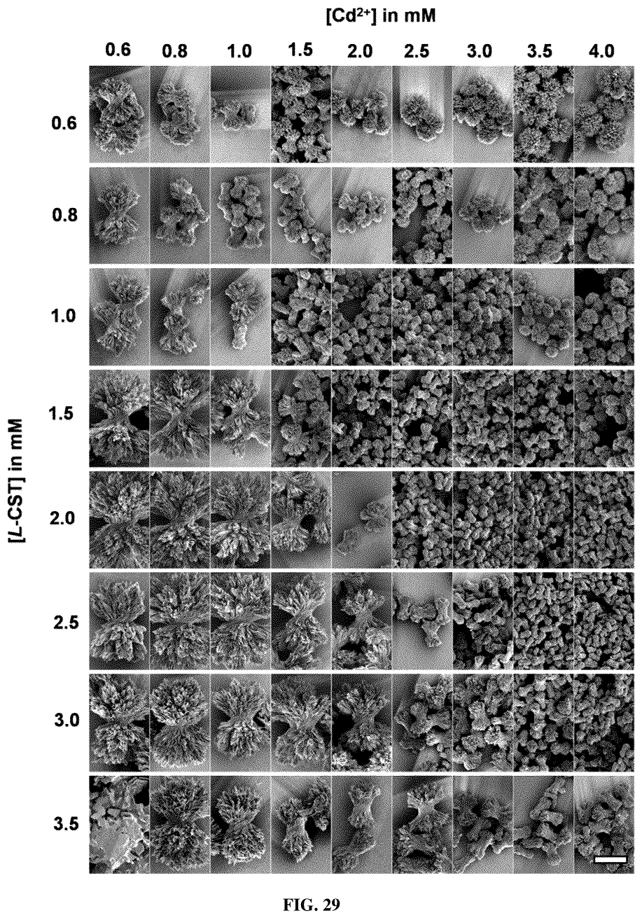

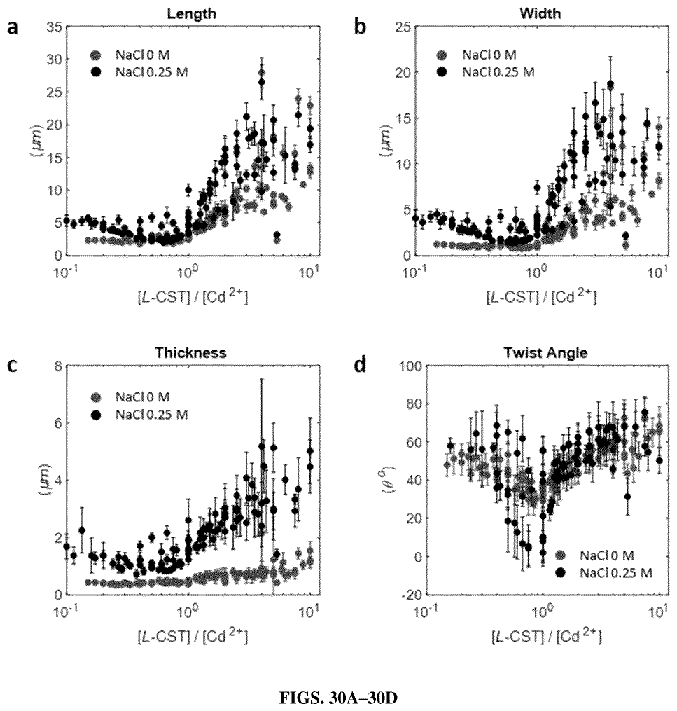

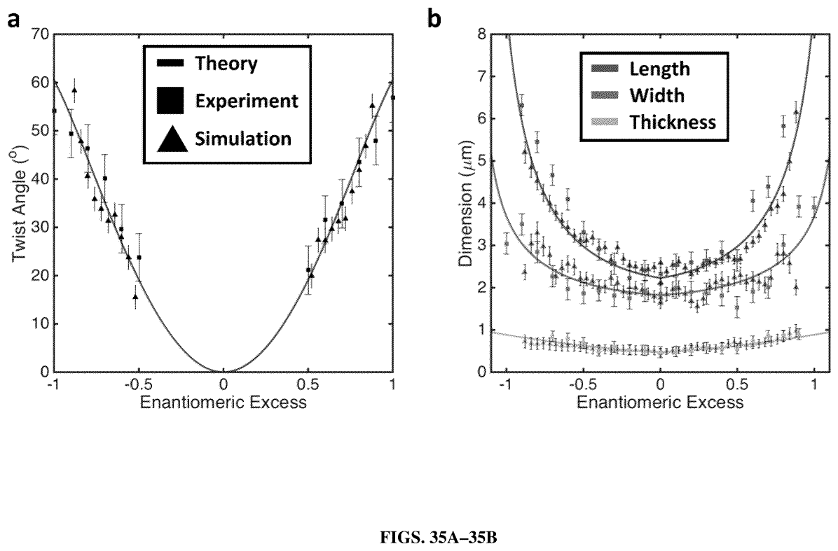

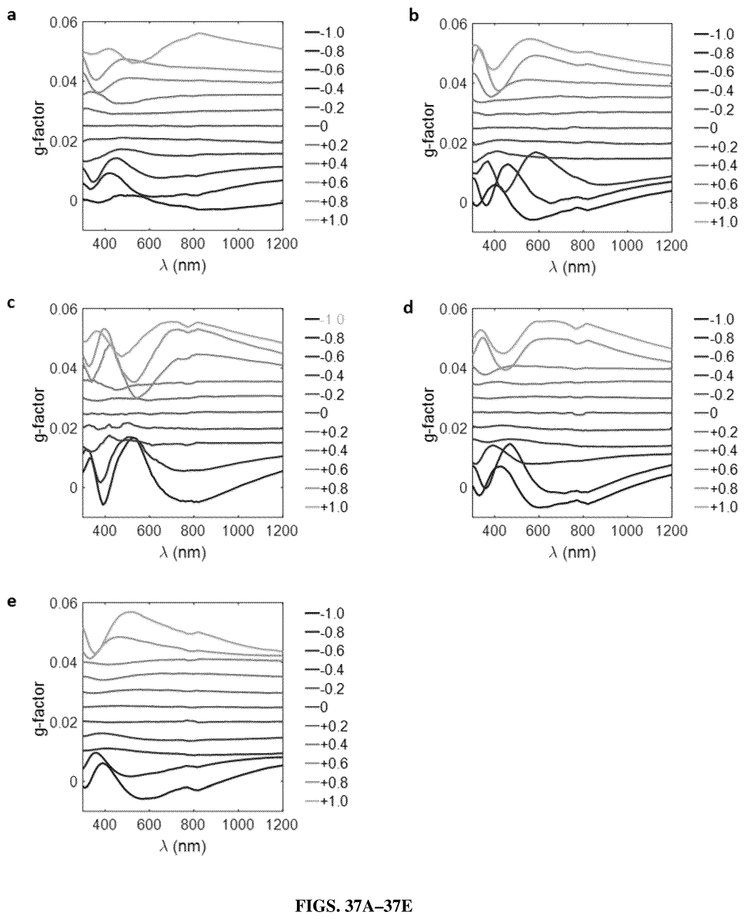

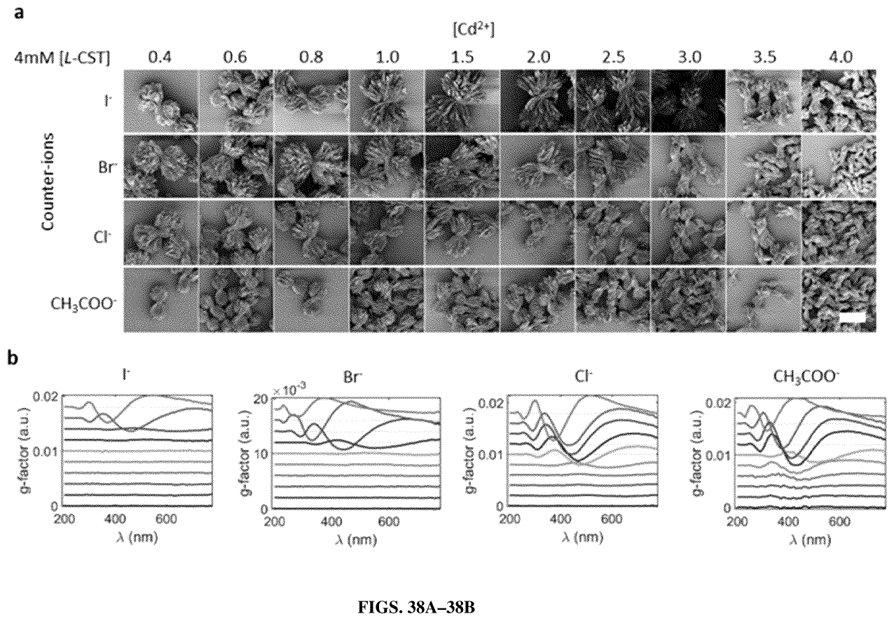

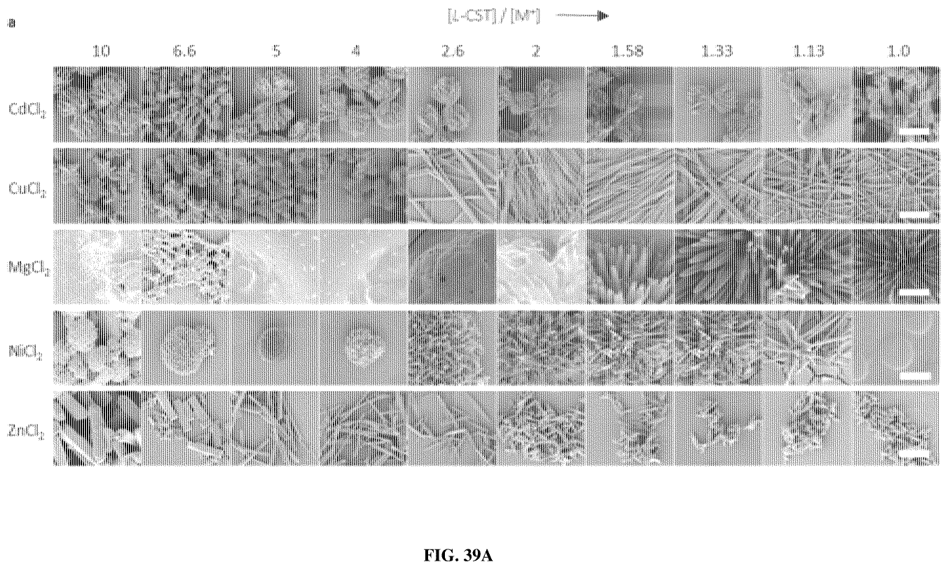

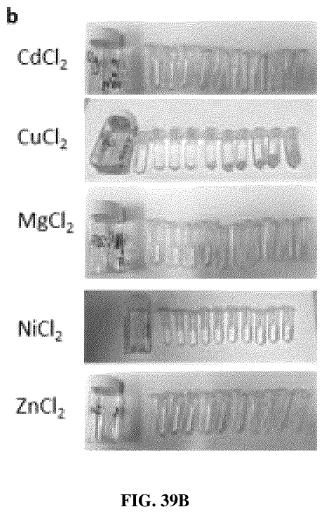

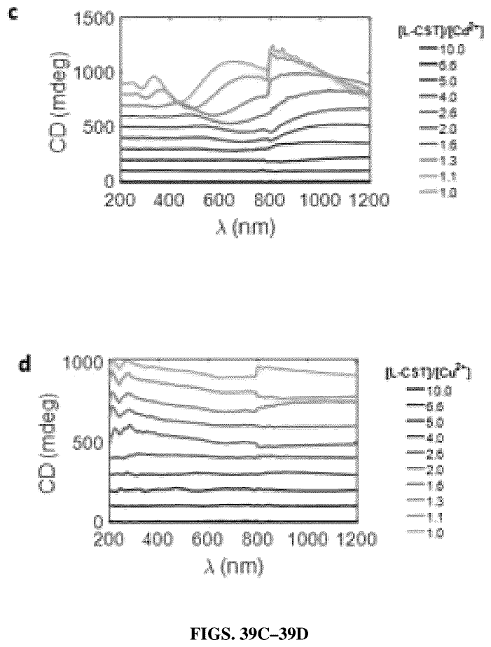

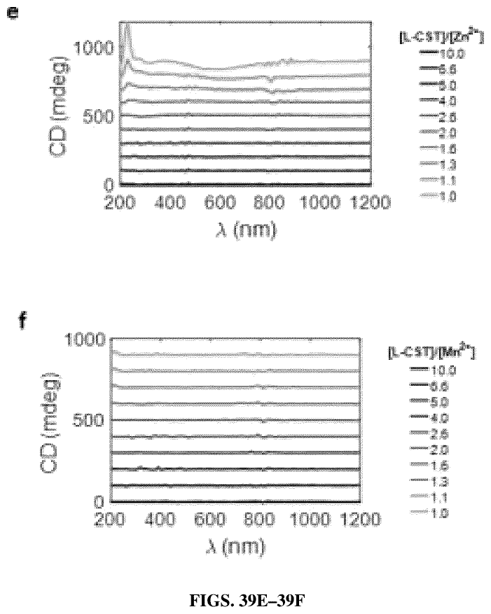

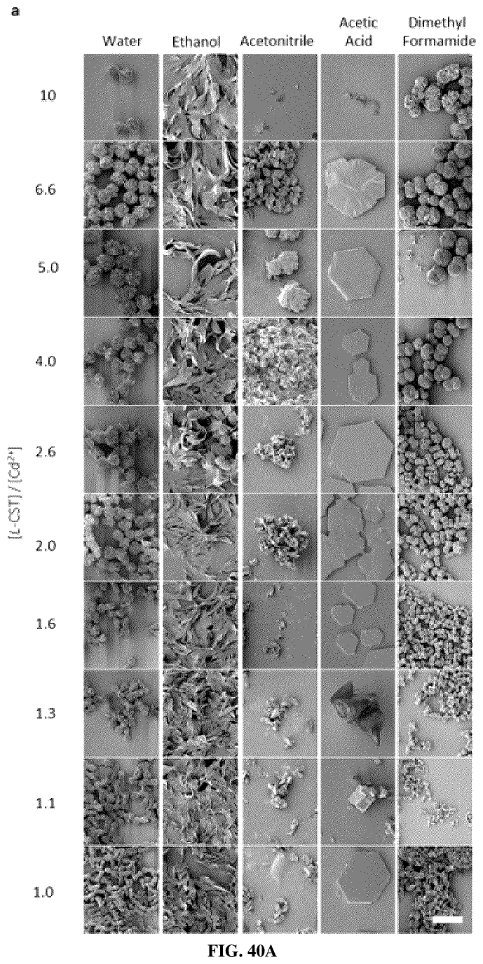

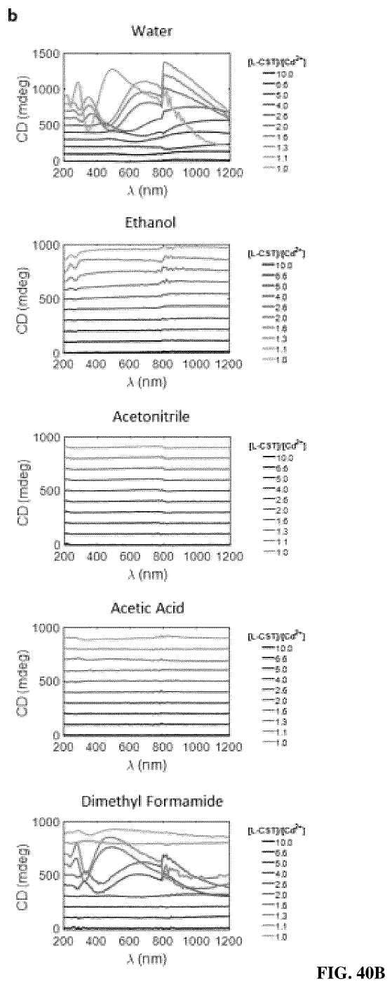

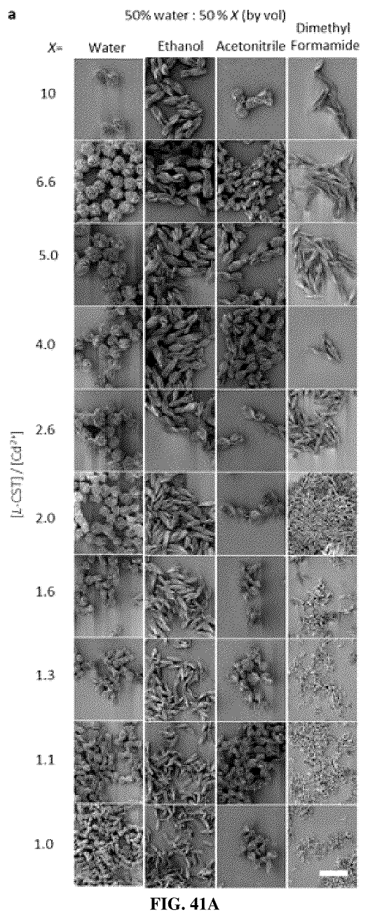

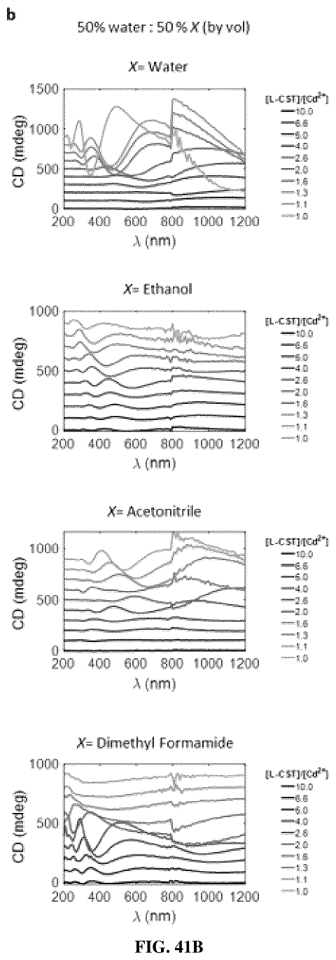

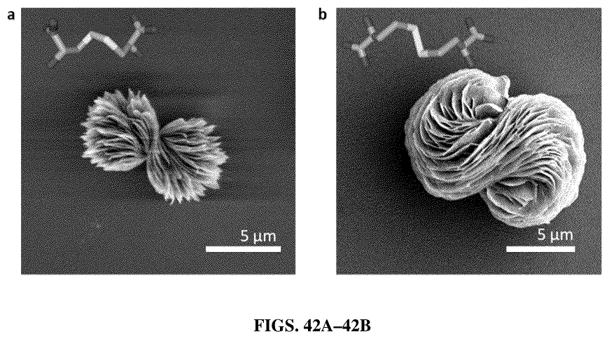

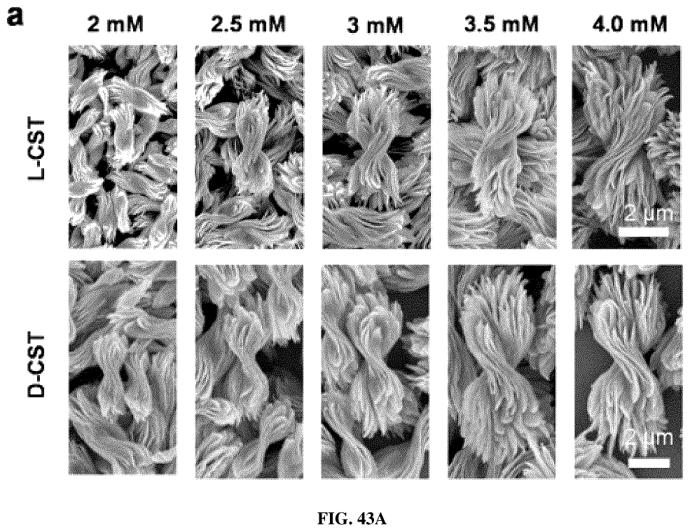

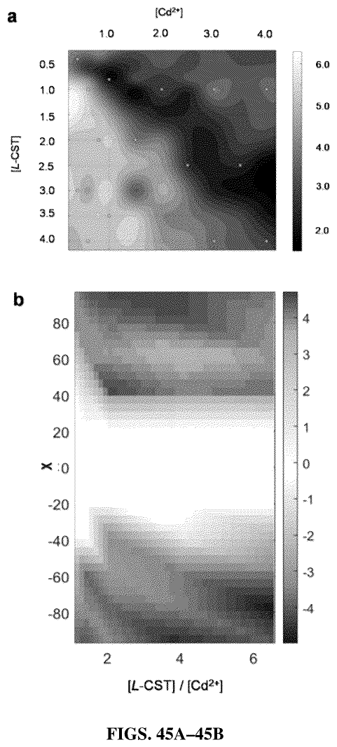

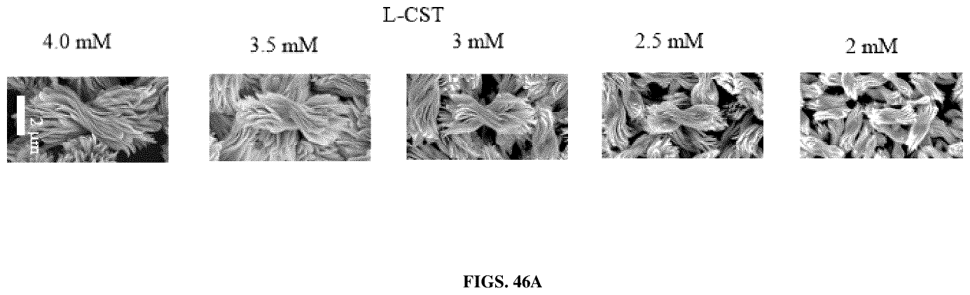

The variability of the twist angle and pitch can serve as a vivid demonstration of the tunable chirality of these particles (FIGS. 45A-45B). The computational model describing the self-assembly of the bowties from nanoclusters quantitatively predicts their geometry also displaying the continuous variation of all the geometrical parameters of the self-assembled particles (FIGS. 3D, 3E, FIGS. 25-35C, FIGS. 37A-41B).

In a mathematically rigorous fashion chirality continuum of bowtie particles can be demonstrated by calculating scale-less chirality measures exemplified by Hausdorff chirality measure, Continuous Symmetry Measure, or Osipov-Pickup-Dunmur chirality measure (OPD) because it changes the sign when switching from left to right enantiomers, which is convenient for description of positive and negative peaks in circular dichroism spectra. OPD for the synthesized particles is found to vary gradually from −4.7 to +4.6 (FIG. 2F) depending on the synthetic conditions.

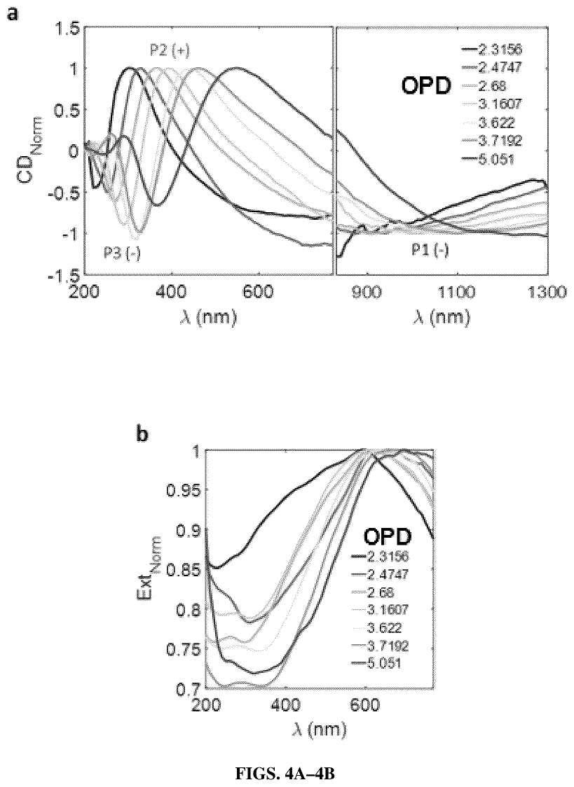

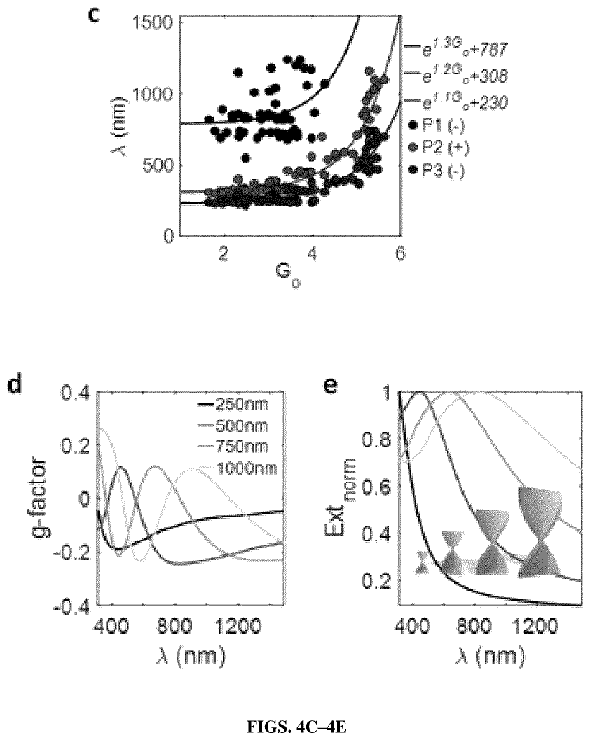

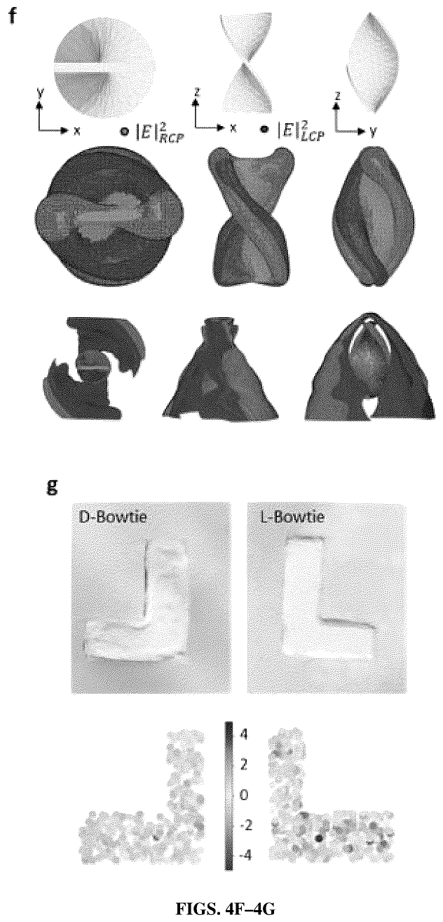

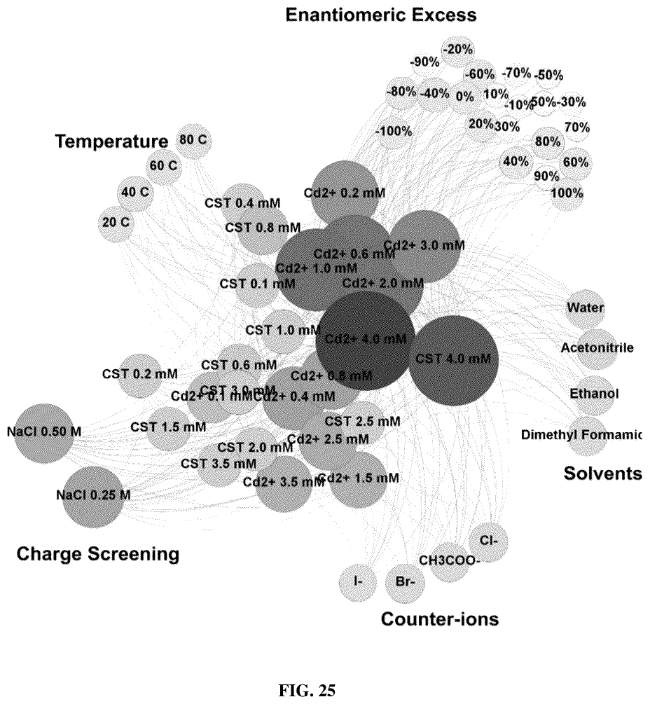

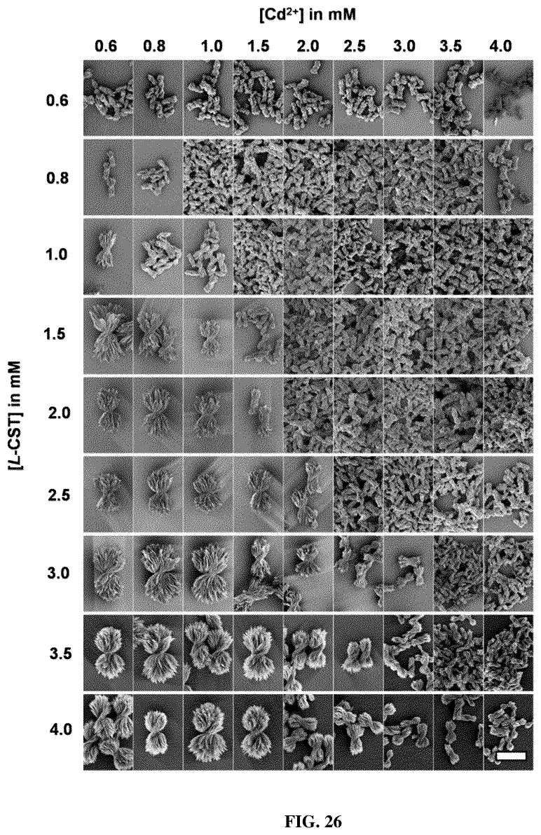

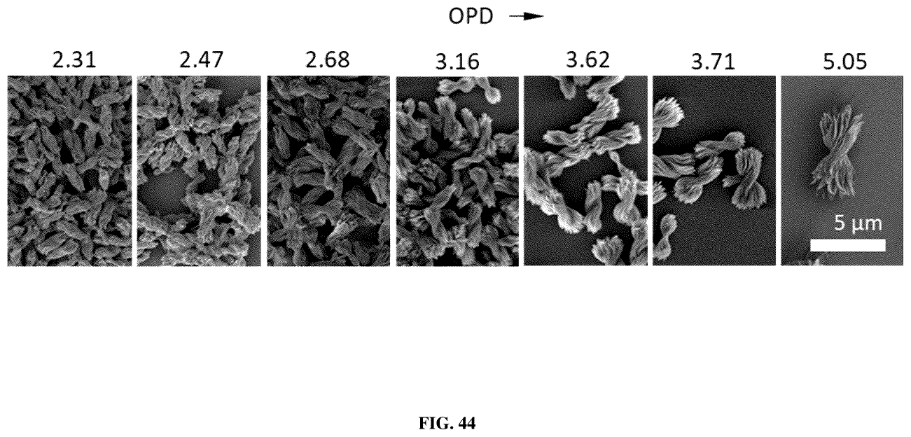

Photonic properties. The nanoscale organization and microscale shape of the particles engenders their strong polarization-dependent interactions with photons in visible and IR parts of the spectrum, which can be appreciated from the large amplitude of the CD peaks in FIG. 1F. P3, P2, and P1 undergo a distinct red shift as the size of the particles increases (FIGS. 4A-4E, FIGS. 26-28). The analysis of the red shift and other spectral trends in FIGS. 1A-1C, FIG. 44 across the parameter space FIGS. 3D, 3E, 25 enables accurate identification of the origin of P3, P2, and P1 peaks located in the visible and near-IR parts of the spectrum.

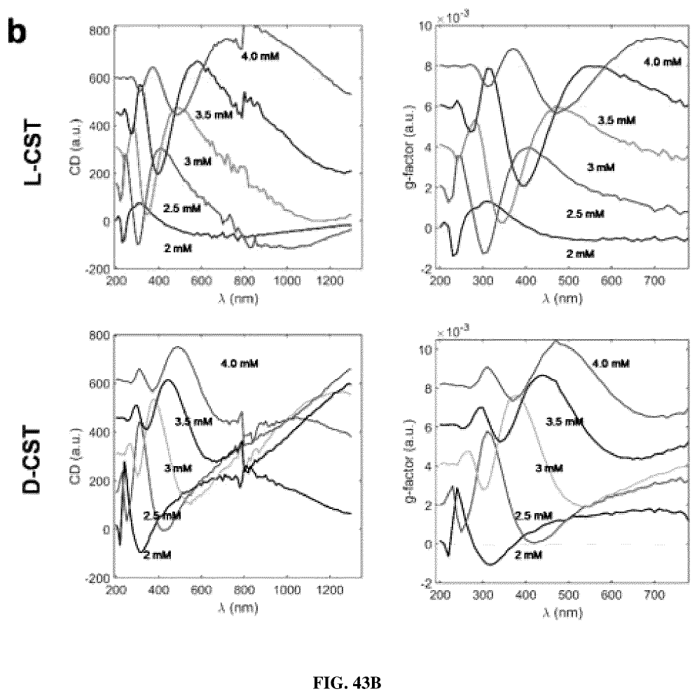

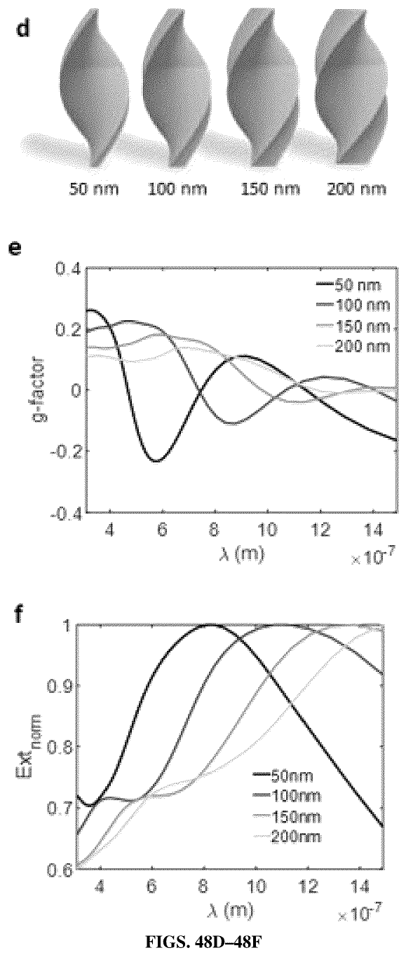

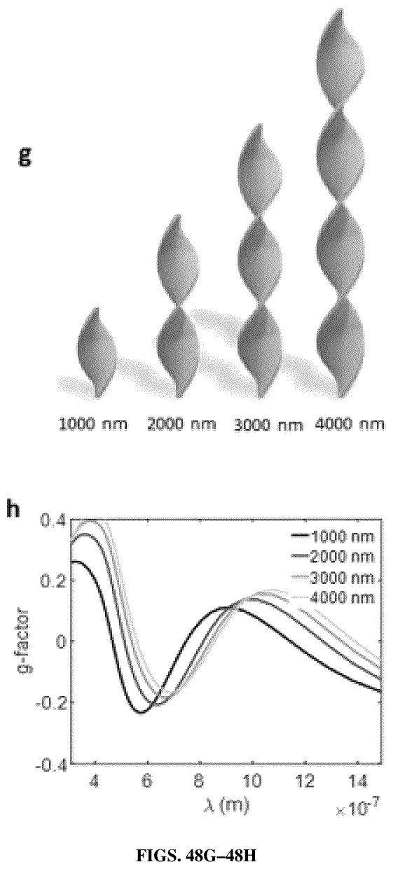

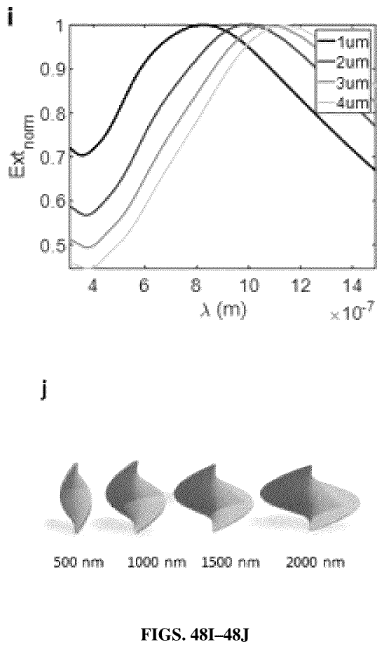

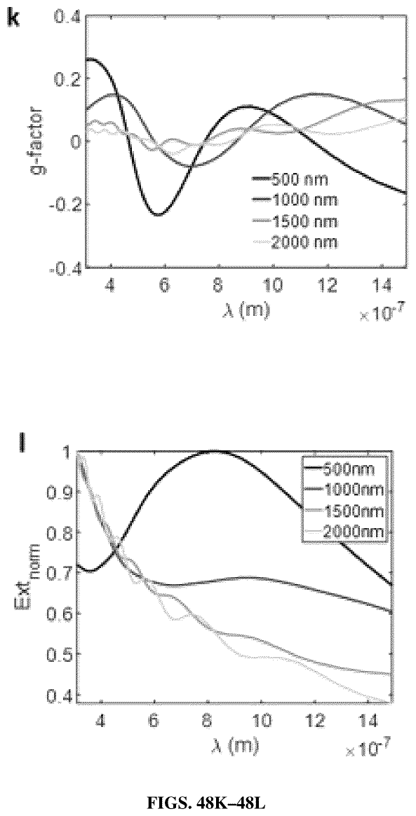

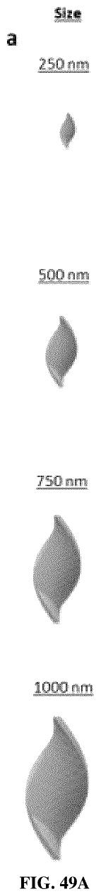

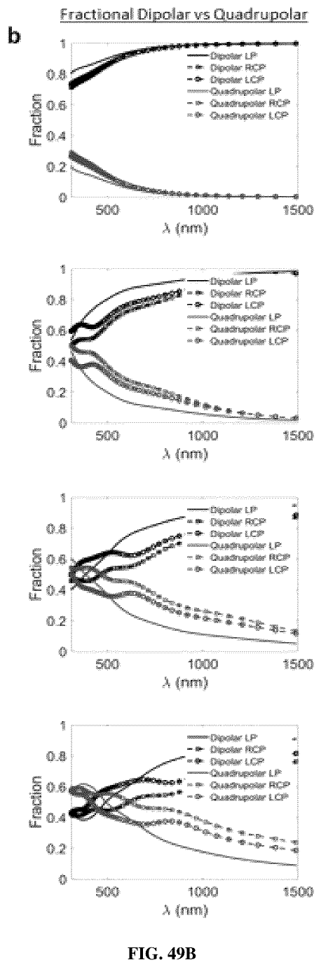

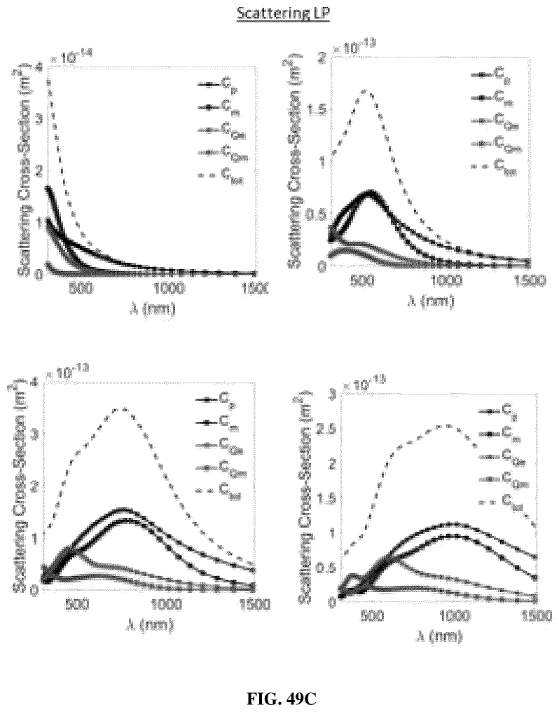

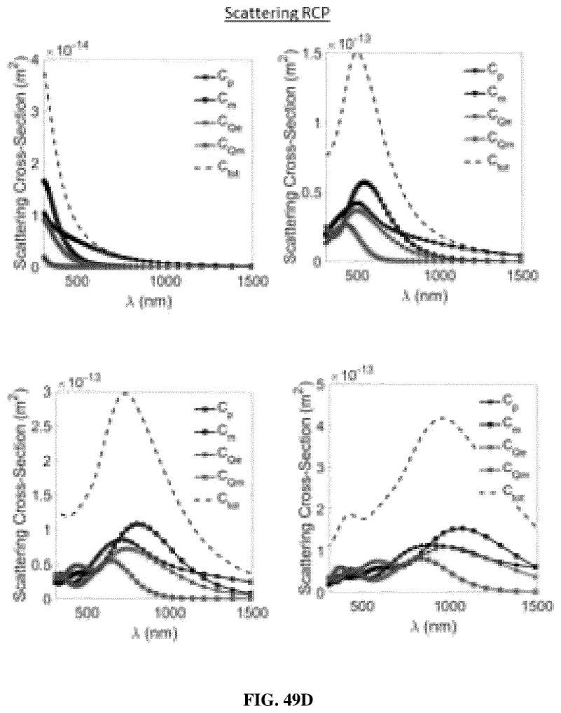

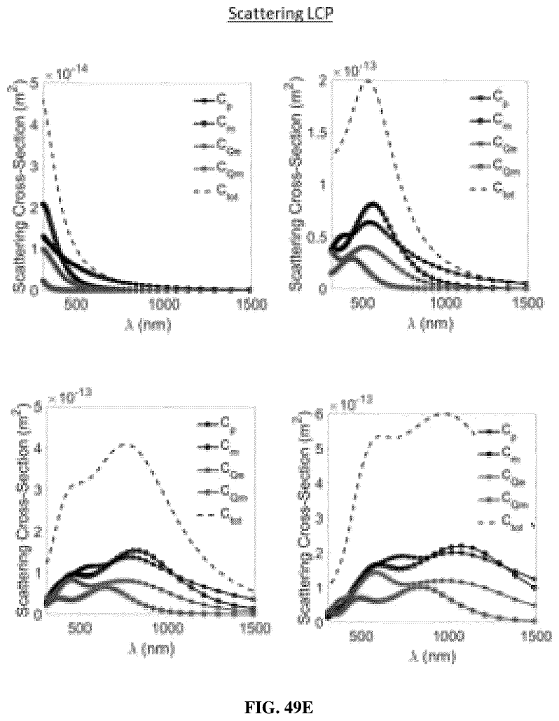

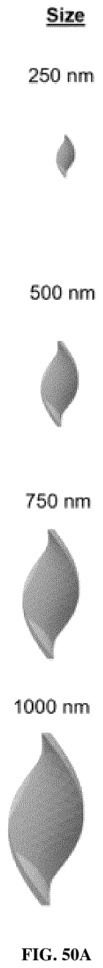

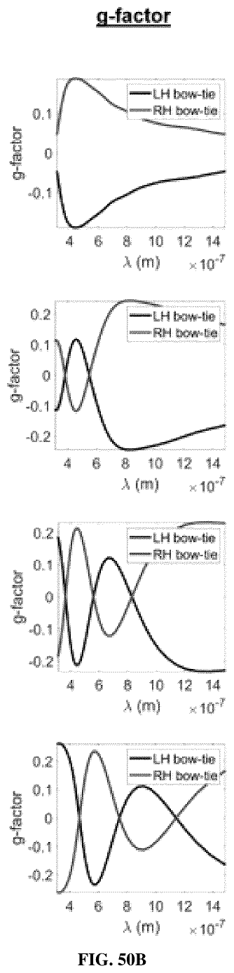

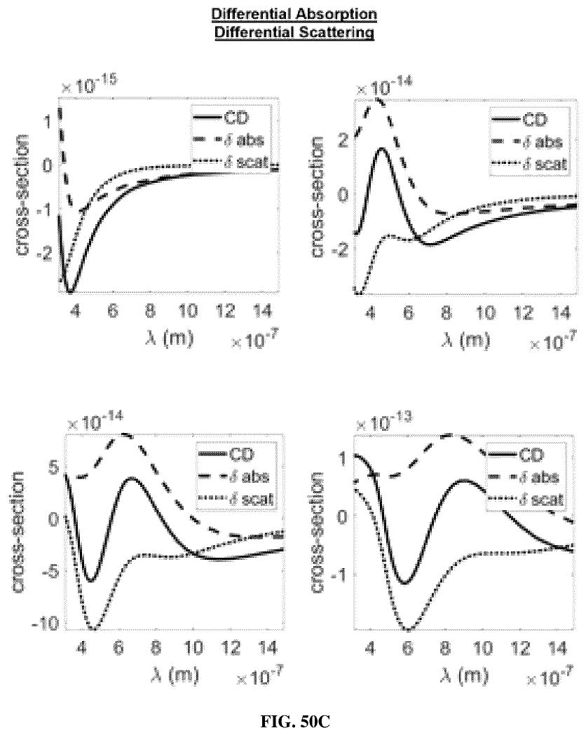

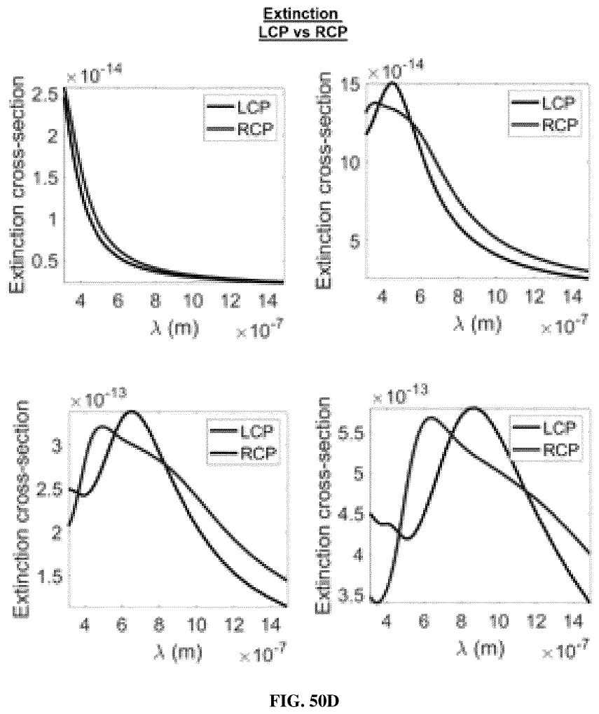

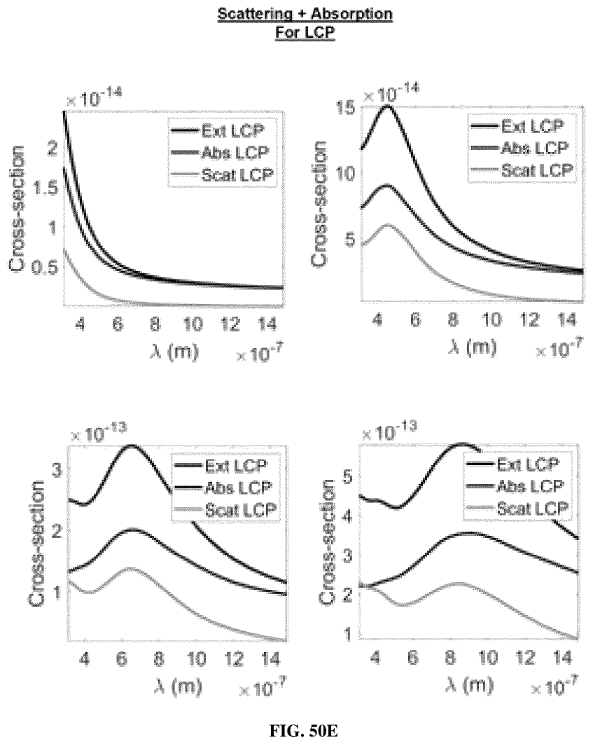

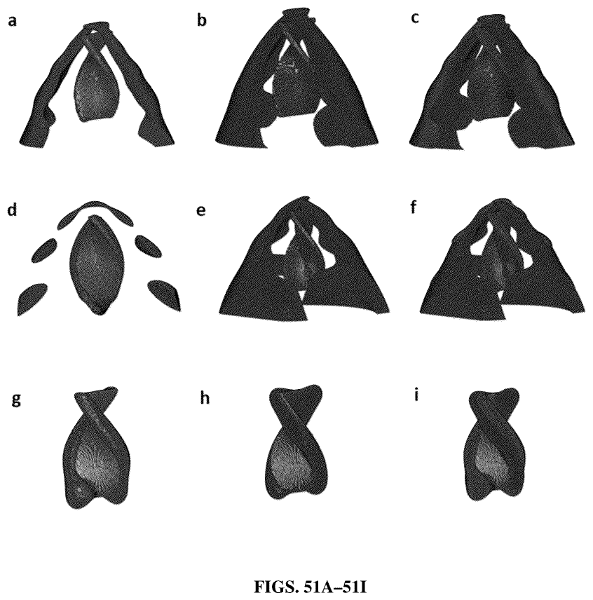

Electromagnetic simulations of their spectroscopic properties are carried out and it was found that the observed spectrum can be rationalized based on computational models with a geometry of single twisted ribbons (FIGS. 3, FIGS. 47A-48L). Taking left-handed models corresponding to bowties made from L-CST as an example, decomposition of the extinction spectra into scattering and absorptive components shows that the positive P3 and P1 peaks in FIG. 1F and FIG. 4A originate from the Mie scattering. The dependence of the peak position matches nearly perfectly the dependences on the particle size observed for the bowties in experiment (FIGS. 3A, 3B, FIGS. 26, 27, 43A-43B, 48A-48L). The negative peak P2 corresponds to the absorption because the absorption intensity for right-handed photons in the visible part of the spectrum is higher than left-handed ones (FIGS. 50A-50E). Further analysis of optical properties scattering processes depending on the electrical and field distributions indicates that P1 and P3 originate from the dipolar and quadrupolar scattering modes (FIGS. 4F, 49A-49E, 51A-51E), respectively. While variable in spectral positions in correspondence with the particle size, the attribution of the peaks remains the same for bowties for all l, w, t, and θ, as can be demonstrated by the calculations for the variety of models (FIGS. 48A-48L).

Organic and biological chemistries do not provide examples of quantifiable property-chirality relationships, despite extensive discussions of their possibility. In case of bowtie particles, a strikingly clear relationship between OPD and the spectral position of P2, P3 peaks is observed (FIG. 4C). The obscure experimental dependence for the P1 peak is attributed to the large error in determination of the peak maximum.

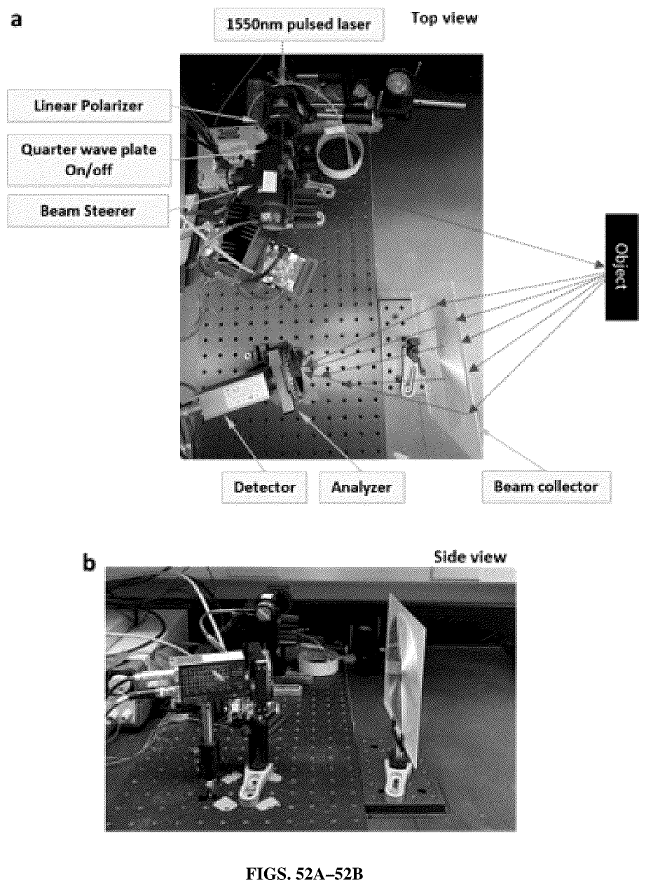

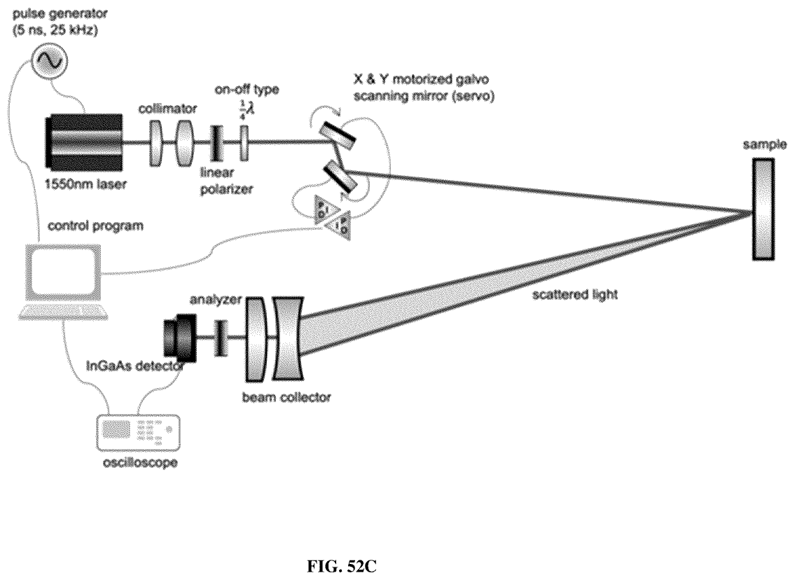

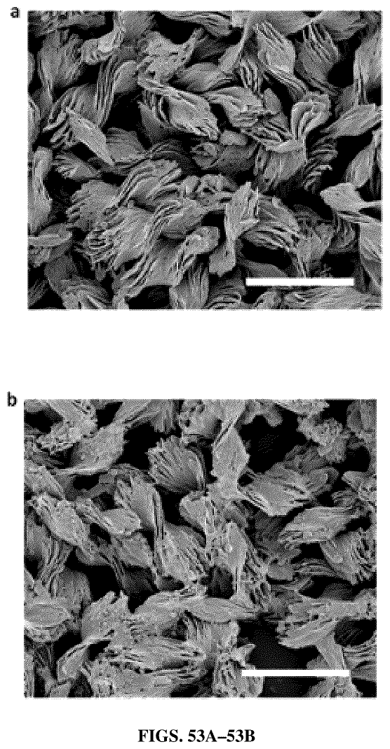

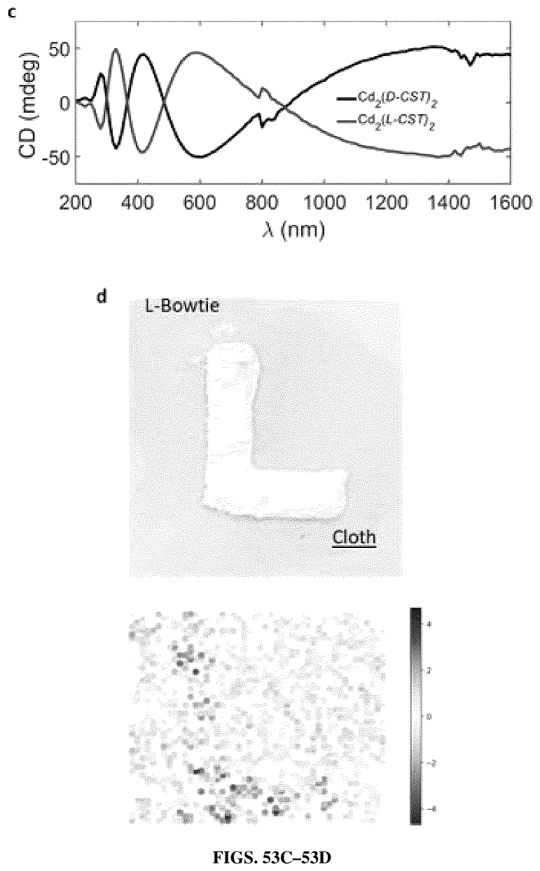

The detailed understanding of the optical nature of the observed polarization peaks, synthetic simplicity of bowties and predictive relationships for optical properties open the door to the engineering of particle-based metasurfaces for photonic technologies. For example, coatings with distinct circular polarization signature (FIGS. 53A-53D) are needed for multiple tasks in machine NIR vision from reduction of glare to continuous perception system calibration with fiducial markers. Thus, bowtie particles with strong circularly polarized P1 peak scattering in NIR part of the spectrum are observed. Their dispersion of the bowtie particles was mixed with poly-acrylic acid (PAA) and printed on glass slides or cotton fabric followed by their imaging using polarization LIDAR operating at 1550 nm (FIGS. 52A-52C). Distinct contrast between prints made from the left- and right-handed bowties can be observed (FIG. 4G). The layer of left-handed bowties can be also identified with high contrast on cotton cloth that give strong right-handed scattering contrast at 1550 nm due to the weaving pattern of the fabric.

Besides LIDAR, the bowtie particles with chirality continuum can also be implemented as fiduciary markers and other labeling methods for polarization cameras and NIR cameras.

In addition to the machine visions of LIDARS for non-biological objects the labeling and polarization analysis can also be used for evaluation of biological objects such as wounds.

The hierarchical assembly ‘imperfect’ nanosheets makes possible a new family of chiroptical materials with a wide range of continuously tunable bowtie geometries. Their chemical and optical properties opens the door for chirality-based engineering of materials for photonic, chemical, biosensing and biomedical technologies taking advantage of tunability of scattering and absorptive peaks. The dispersibility of the bowtie particles creates a possibility for printable metasurfaces, which can simplify their scalable manufacturing and utilization.

### EXAMPLES

Electron microscopy. Scanning electron microscopy (SEM) samples were prepared by drop-casting 5 μl of aqueous dispersion on 1 cm×1 cm silicon wafers (TedPella), following by drying at room temperature. This was followed by sputter coating a 5-10 nm film of gold on the wafer to avoid charging due to the electron beam. SEM measurements were performed in FEI Nova NanoLab Dual Beam SEM and FEI Helios Nanolab at 5 kV accelerating voltage and 0.4 nA beam current under secondary electron detection mode.

TEM samples were prepared by drop-casting 10 μl of aqueous solution on to a copper grid coated with holey carbon supported by a continuous carbon film (TedPella 01824). Bright-field TEM was performed on JEOL 2010 operating at 300 kV accelerating voltage and acquisition were done using Gatan OneView camera. ADF-STEM and STEM-EDX measurements were performed on cold-FEG JEOL 3100R05 with Cs aberration correction operating at 300 keV. A HAADF detector was to acquire Z-contrast images where the intensity is proportional to the atomic number of the column over which the electron probe is placed. Diffraction experiments were acquired using Thermo Fisher Talos F200X operated at 200 keV equipped with a Gatan One View camera. The Elsa (698) Gatan Cryo Holder cooled specimens down to ˜93 K for low-temperature measurements.

TEM Tomography—The synthesized bowtie particles were dispersed in water and drop cast using a micropipette onto a 3 mm copper TEM grid dried at room temperature. The TEM grid was an ultrathin (3 nm) carbon film with a large hexagonal mesh (100) to provide high specimen tilts without beam shadowing (Electron Microscopy Sciences, Hatfield, PA, USA). The tomographic tilt series were acquired using a Thermo Fisher Talos F200X operated at 200 keV with a 10.5 mrad semi-convergence angle using a 36 mrad and 165 mrad inner and outer semi-collection angles for the annular dark field (ADF) detector. The right-handed bowtie particles was acquired over a tilt range of −72° to +73° and left-handed bowtie particles specimen was acquired over a tilt range of −75° to +71° both with a +1° tilt increment. At each angle, annular dark-field images of size 1024×1024 pixels were recorded with a dwell time of 4 us and pixel size of 4.94 nm. The tomograms were reconstructed with the additive simultaneous iterative reconstruction technique for 150 iterations. The three-dimensional reconstructions were visualized by tomviz

X-Ray Diffraction. Synchrotron X-ray powder diffraction data (λ=0.45192 Å) was collected at Beamline 17-BM at the Advanced Photon Source at the Argonne National lab. Freeze dried powder sample of L-/D-/rac-bowties was loaded in a 1.0 mm diameter Kapton capillary tube, which was then measured using a VAREX XRD 4343CT amorphous silicon area detector in the Debye-Scherrer geometry.

Measurements of particle geometry. Length (l), width (w), thickness (h) and angle of twist (θ) as defined in FIG. 3C were measured using Jann5s/measure tool from Matlab central. A minimum of 10 measurements was done for each parameter and standard deviation (σ) was calculated and represented as percentage variation by following equation

$$error = \frac{{σ}_{x}}{\underline{x}} \times 100 ,$$

where x=l, w, h, θ and x is the mean of corresponding values.

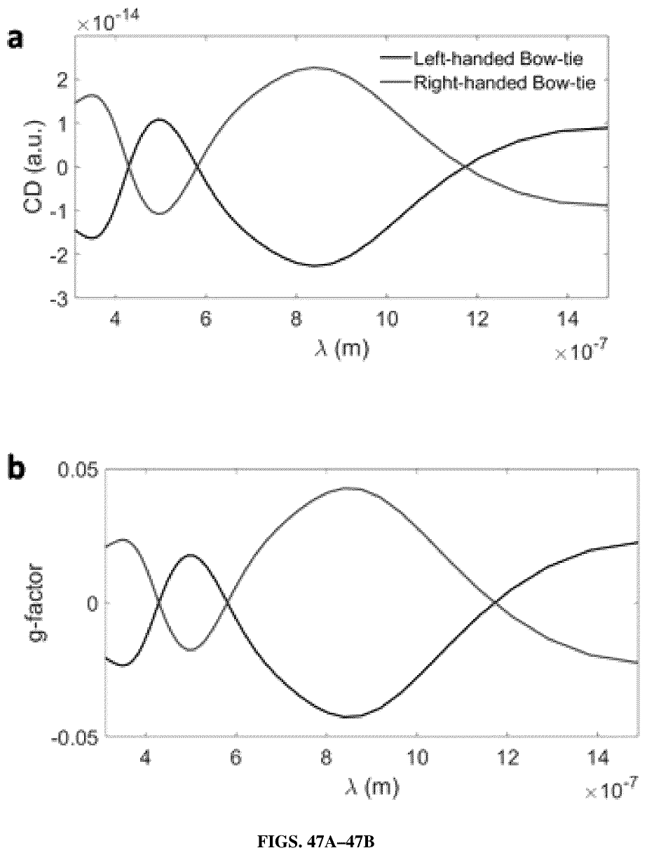

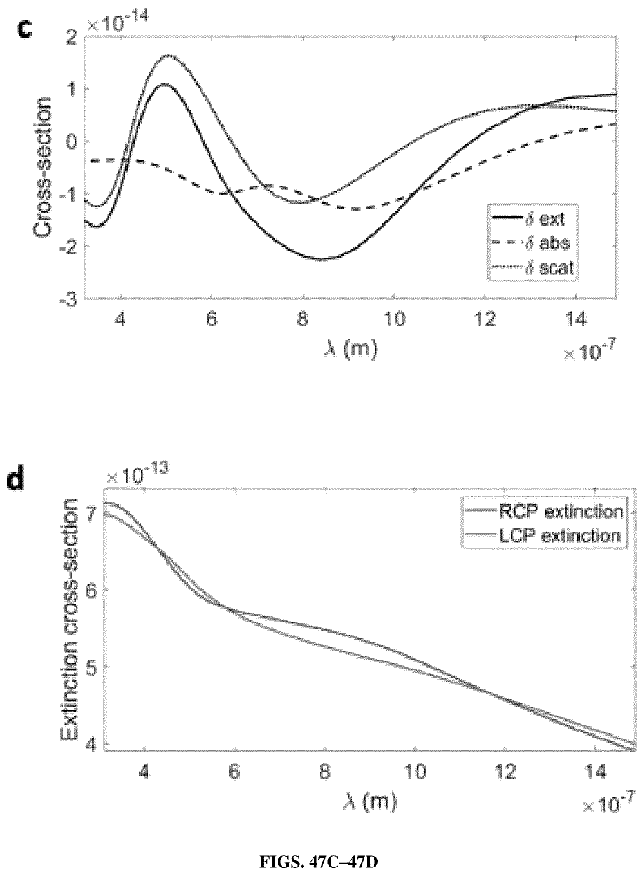

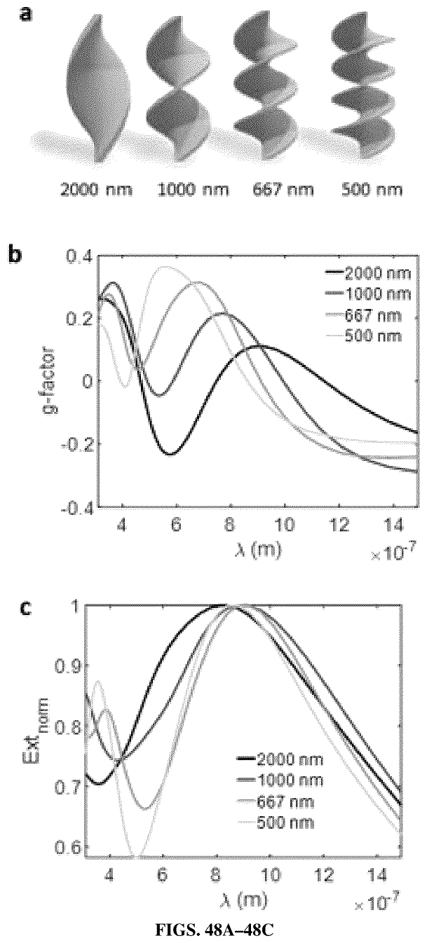

Finite-difference time-domain (FDTD) simulations of optical properties—The CD and g-factor spectra were calculated using a commercial FDTD software package (Lumerical Solutions Inc.; www.lumerical.com/tcad-products/fdtd/). Total-field scattered-field (TFSF) sources are used that surrounded the structure being modeled. CPL was generated by positioning two TFSF sources along the same forward axis at a 90° angle and with a phase difference of either −90° for photons with left-handed polarization (LCP) or 90° for photons with right-handed polarization (RCP). Two analysis groups using box power monitors monitored the absorption and scattering cross sections (extinction is the sum of absorption and scattering). The FDTD simulation region was defined by a larger box monitor with a stretched-coordinate perfectly matched layer and non-uniform mesh type. Frequency profile monitors were inserted in the total field region to calculate electric field distribution in 3D. The refractive index for water was 1.33. Convergence tests with different mesh sizes were performed to determine the best balance between computational time restraints and simulation accuracy. Simulations were carried out for the bowties orientated with their long axis being parallel with the propagation direction (k-vector) of photons. 10-nm and 1 nm mesh size produced similar CD spectra; therefore, 10-nm mesh size is used for bowtie simulations presented in FIGS. 47A-50E.

Chiroptical spectroscopy. Circular dichroism measurements in UV-vis and NIR range were performed using JASCO J-1700 equipped with one PMT detector in 200-800 nm range and two InGaAs NIR detectors in 800-1600 nm range. Typical scanning parameters were as follows: scanning speed, 500 nm/min; data pitch, 0.1 nm; bandwidth, 5 nm (NIR bandwidth, 10 nm), digital integration time, 0.25 s; and one accumulation. The anisotropy g-factor was calculated according to the equation

$$g = \frac{CD}{32 , 980 \times abs} ,$$

where CD Is the signal obtained from CD/DC channel, abs is the absorbance calculated from ABS and DC channels of the spectrophotometer. CD spectra were stopped at wavelengths where the HT voltage was greater than 800 V to avoid artifacts during acquisition.

Terahertz circular dichroism spectroscopy (TCD). Terahertz (THz) time-domain polarimetry system based on three linear polarizers was used to measure absorption coefficients and THz circular dichroism spectra. Calculations of Stokes parameters are based on the Ex and Ey, the electric field in x and y directions, respectively. Absorption coefficients were extracted from the equations used in ref. after retrieval of phase from the transmission data. To eliminate linear birefringence effects from samples, highly concentrated mixture of bowtie powders with mineral oil was used. The reference sample is a quartz sandwich cell filled with mineral oil.

Vibrational circular dichroism spectroscopy. VCD measurements were performed on freeze dried bowtie samples dispersed in heavy water (D₂O) at 33 mg/ml concentration. A 100 μl drop was sandwiched between two BaF₂ crystals separated by 50 μm Teflon spacer. MCT-V detector was used to acquire IR and VCD data in the range 2000-850 cm⁻¹ with a resolution of 4 cm⁻¹ and a total of 100 and 500 accumulations respectively. The sandwiched dispersion between BaF₂ crystals was rotated along a axis coinciding with the direction of the beam at a constant speed to avoid settling of particles. Corresponding IR and VCD were plotted as A and

$$\frac{Δ A}{A}$$

respectively with exclusion of 1300 to 1100 cm⁻¹ range that corresponds to strong absorption from D₂O.

### Methods

### XRD Data Acquisition.

Preliminary structure evaluation was done using powder X-ray diffraction data acquired from a Rigaku Smartlab diffractometer with a source of Cu-Kα at 40 kV voltage and 44 mA current (λ=1.54059 Å). The optics included a K-beta filter and Soller slit. The diffractometer was operated in Bragg-Bentano configuration, and the signal was collected on Scintillation Counter (SC-70), D/tex ultra 250 high speed silicon strip 1D. Data was acquired from 5° to 75° with a step size of 0.01°. Synchrotron X-ray powder diffraction data (λ=0.45192 Å) was collected at Beamline 17-BM at the Advanced Photon Source at the Argonne National lab. Freeze dried powder sample of L-/D-/rac-bowties was loaded in a 1.0 mm diameter Kapton capillary tube, which was then measured using a VAREX XRD 4343CT amorphous silicon area detector in the Debye-Scherrer geometry. The resulting 2D diffraction pattern was integrated using the GSAS-II software into an 1D pattern of intensity versus 20, which was then used for structure solution and refinement.

### XRD Structure Solution and Refinement

Structure solution was performed with the TOPAS software on the synchrotron powder XRD data. A total of 31 low angle peaks were used for indexing. The result indicated an orthorhombic unit cell with three edges of around 20.48 Å, 9.94 Å and 5.05 Å. Considering the chirality of the bowtie morphology of the material observed under electron microscope, the spaces group should be one of the Sohncke groups that have only symmetry operations of first kind. The extinction conditions of the space group P2₁2₁2₁ shows the best match with the observed PXRD data, though P2₁2₁2 was also tried in the structure solution process. Unit cell parameters, sample displacement, background and profile parameters were refined by Pawley refinement and fixed in the subsequent structure solution process, where the simulated annealing method built in TOPAS was used. The L-CST was modeled as rigid body with flexibilities on bond angles and torsion angles. Cd was treated as a free atom. Chemical analysis based on the EDX result and the titration measurements pointed to a Cd/S ratio of 1 to 2, equal to 1 Cd per L-CST molecule. Considering the cell volume, 1 Cd atom and 1 L-CST were employed per asymmetric unit. Hydrogen atoms were not modeled. The correct structure solution did not appear until after more than 50 attempts, mainly due to the low resolution of the data, which has a minimum d-space of 1.6 Å and shows severe peak overlap at below 2.5 Å as a result of the nanosized particles of the sample, as well as the low sensitivity of the data to different configurations of L-CST. After the correct structure model emerged, Rietveld refinement was performed. The atomic displacement parameter (ADP) of all the atoms in L-CST were constraint to be the same, while the ADP of Cd was refined independently. Other than the ADPs, fractional coordinates of the Cd site, translation and rotation of the L-CST rigid body, bond angles and torsion angles within the rigid body, unit cell parameters, the sample displacement parameter and the background were also refined. The background was modeled by Chebyshev polynomial of 3 coefficients. In the final cycle of refinement generating the crystallographic information file (CIF), the two ADP parameters, Cd atomic fractional coordinates, the L-CST rigid body translation, cell parameters and sample displacement were refined. Including other parameters in the final cycle did not visibly change the structure or the quality of the fitting, but greatly enlarged calculated errors of fractional coordinates of many atoms. This is due to the resolution of the diffraction data and ultimately attributed to the nanostructured nature of the material.

### LIDAR Measurements

The customized bench-top polarization LIDAR system was used for the measurement of the polarization effect of scattering light from the deposited L and D-type bowtie particles. As shown in the scheme, 5 ns and 25 kHz pulsed 1550 nm laser (Bktel) was activated by pulse generator (Quantum composer 9200) and powered by 5 V/3 A power supply. The collimated laser beam can be modulated to any polarization state, linear to circular, by passing through the combination of a linear polarizer and a quarter wave plate. The polarized incident beam is directed by motorized X-Y galvo mirror steerer (Thorlabs) to scan the sample in the X-Y space. The servomotor is controlled by an NI DAQ instrument with 10 points/sec. The scattered light from the sample is collected with the large beam collecting lens system and detected by 400 MHZ InGaAs photodetector (Femto) connected with the oscilloscope (picoscope) to capture the intensity and time of flight of pulse that hits the samples to capture the full 3D point cloud data. The pulse generator, DAQ motor controller, and intensity detecting oscilloscope are synchronized using in-lab written MATLAB code to save the XYZ 3D-coordinates of scanned area and its polarized intensity at each point.

Firstly, to measure the linear dichroism effect of the sample from the scattered light at 1550 nm, the linear polarizer at the incident beam needs to be set to 0° and 90° leaving the quarter wave plate at “off” state. At every scan point, the value of LD effect would be measured to see if there is linear difference in L- and D-bowties.

$$Linear Dichroism ( LD ) scattering effect = Intensity ( 0 ° ) - Intensity ( 90 ° )$$

Similarly, the circular dichroism would be measured by the following equation and the linear polarizer at the incident beam will be set to 0° and 90°, for the LCP and RCP respectively, leaving the quarter wave plate at “on” state (fast axis is 45° off from the 0°).

$$Circular Dichroism ( CD ) scattering effect = Intensity ( LCP ) - Intensity ( RCP )$$

If the sample is L-type, it should absorb LCP light more than RCP, as a result, RCP scattering would be dominant (CD scattering effect<0). For the D-type sample, vice versa, and the CD scattering effect>0.

### Simulation of Bowtie Growth

Coarse-Grained Modeling of Bowtie Growth. Structural analysis using XRD data suggests multiple possible solutions (i.e. structures) that can fit the crystallography results. As such, rather than directly mapping one particular structure to a coarse-grained model, relevant features are picked that are consistent across the possible solutions to construct a generalized building block that can reproduce the assembly behavior in the Monte Carlo growth simulations. The key features chosen to adapt are (FIG. 15A): 1). Building blocks are rectangular prism-like and flat with rough dimensions of 20×10×5 as suggested from XRD. 2). H-bonding sites decorate the two largest and two smallest facets to reflect the bonding between the —COOH and —NH₂ groups. 3). The two intermediate facets exhibit specific, directional interaction to capture the disulfide bridge formation intrinsic to the CST used in the experimental system. These interactions sites only interact with themselves. For ease of notation, these sites are referred to as sulfur linkage sites. 4). Building block exhibit a slight shear on one facet to enforce chirality of L-CST vs R-CST. 5). Isotropic charge-charge repulsion between Cd²⁺ atoms embedded inside the rectangular building block. Each building block has a scaled net charge of +1 to reflect a net charge on building blocks indicated by experiemtanl zeta-potential measurements. This choice additionally builds in charge-driven, self-limiting behaviors. 6). All other surface sites not defined by h-bonding or sulfur linkage exhibit steric repulsion.

Computing potentials of mean force (PMF) between coarsed grained building blocks reveal that the proposed model exhibit the features necessary to capture the heirarchy of structural formation observed in experiments: that is, chains assembling into nanosheets that then stack to ultimately form the observed bowtie structures (FIGS. 2G, 2H). PMFs computed for a mix of L-CST and R-CST reveals a reduction of tendency in hierarchical structural formation, driving the transition from bowtie to pancake structure. The computed PMFs are employed to perform Monte Carlo growth simulations to confirm the formation of both bowtie and pancake morphologies (FIGS. 2I, 2J).

Modeling of Bowtie assembly. For the system of interest, there are three possible choices for CST enantiomers in the helical chains from Cd₂CST₂ units producing one racemic and two homochiral versions that can be abbreviated as LCD, LCL, and DCD. LCL and DCD are equal in energy when they do not interact with other chiral species. However, LCD differs from LCL (or DCD) by an energy quantity AU. Thus one can describe the probability of their formation in the mixture of CST enantiomers with different χ by writing the partition function Z as Z=1+e^{-βΔU}, where β=1/k_{b}T. The probability P_{LCD} of forming the LCD nanocluster is directly related to enantiomeric excess of CST, χ, used in experiments. Using classical statistical mechanics, the Boltzmann weighted energy difference can be equated to P_{LCD} to give

$$1 - \frac{1 + χ}{2 + χ} = \frac{{e}^{- β Δ U}}{1 + {e}^{- β Δ U}} \qquad ( 1 )$$

Solving for ΔU gives the energy difference of LCD relative to LCL (or DCD) explicitly in terms of χ to be βΔU˜ln(1+χ). The average twist angle, given an excess fraction of enantiomer, can then be determined as [symbol] cos(θ(χ)) [symbol] =∫cos(θ(χ))P(θ(χ))dθ. For this system, there are only three possible choices for CST enantiomers—LCD, LCL, and DCD—enabling us to convert the integral to a sum over all 3 enantiomeric types.

$$〈 cos ( θ ( χ ) ) 〉 = {\sum }_{i ϵ \{ LCL , LCD , DCD \}} cos ( {θ}_{i} ( χ ) ) P ( {θ}_{i} ( χ ) ) \qquad ( 2 )$$

Plugging in P(θ(χ)) for each respective state and noting that P_{LCL}=P_{DCD}, provides

$$〈 cos ( θ ( χ ) ) 〉 = {Z}^{- 1} \{ {( 1 + χ )}^{- 1} cos ( {θ}_{LCD} ( χ ) ) + {2}^{- 1} [ cos ( {θ}_{LCL} ( χ ) ) + cos ( {θ}_{DCD} ( χ ) ) ] \} \qquad ( 3 )$$

Let the angle between LCL be θ_{c} and noting that LCL and DCD are mirror images (i.e., θ_{LCL}=−θ_{DCD}) gives

$$〈 cos ( θ ( χ ) ) 〉 = {Z}^{- 1} \{ {( 1 + χ )}^{- 1} cos ( {θ}_{LCD} ( χ ) ) + {χcosθ}_{c} \} \qquad ( 4 )$$

Additionally, the angle for LCD is approximated to be small (to reflect the “pancake” motif). Perturbation of cos(θ_{LCD} (χ)) about a small θ_{LCD} and taking the first term gives

$$〈 cos ( θ ( χ ) ) 〉 = {Z}^{- 1} \{ {( 1 + χ )}^{- 1} + {χcosθ}_{c} \} \qquad ( 5 )$$

From earlier, Z=[2+χ][1+χ]⁻¹ is known, plugging Z into Eq. 5 gives

$$〈 cos ( θ ( χ ) ) 〉 = \frac{χ ( 1 + χ ) cos {θ}_{c} + 1}{2 + χ} \qquad ( 6 )$$

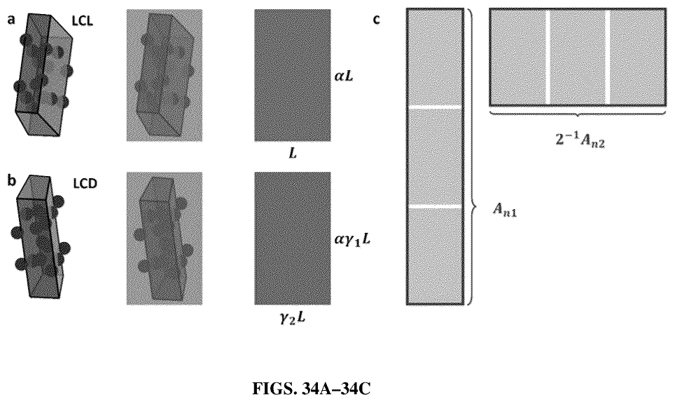

Eq. 6 is employed to predict the relevant dimensions of the resulting bowtie particles. The growth directions reflect end-to-end or top-bottom stacking of the coarse-grained building blocks. As such, a scaling model approach to computing the gain in surface area as a function of increasing number of nanoclusters can be written as follows. (FIGS. 34A-34C)

$${A}_{n 1} ∼ {L}^{2} [ \frac{1 + ( n - 1 ) ( {P}_{1} {γ}_{2} + {P}_{2} )}{n} + 2 α + 2 α ( n - 1 ) ( {P}_{1} {γ}_{1} {y}_{2} + {P}_{2} ) + ( n - 1 ) {{P}_{1} ( {γ}_{2} - 1 )}^{2} ] \qquad ( 7 a )$$

$${A}_{n 2} ∼ {L}^{2} [ 1 + ( n - 1 ) ( {P}_{1} {γ}_{1}^{2} + {P}_{2} ) + α + α n ( {P}_{1} {γ}_{1} {y}_{2} + {P}_{2} ) + α ( n - 1 ) {{P}_{1} ( {γ}_{2} - 1 )}^{2} ] \qquad ( 7 b )$$

where Aₙ₁ and Aₙ₂ are the areas for edge-edge and top-bottom stacking directions respectively. L is the characteristic length of the nanocluster along its long axis, a is the aspect ratio measuring the amount of nanocluster elongation, n is the number of nanoclusters in the assembly, and γ₁ and γ₂ are the local curvature of the nanoclusters along the long and short direction, respectively, that enables generalization of this model to differently shaped building blocks. P₁ and P₂ are defined as

$${P}_{1} = P ( LCL ) P ( LCD ) = {( 2 Z )}^{- 1} {( 2 + χ )}^{- 1} \qquad ( 8 a )$$

$${P}_{2} = P ( LCL ) P ( LCL ) = {( 2 Z )}^{- 2} \qquad ( 8 b )$$

P₁ and P₂ can thus be interpreted as the probability of an LCL (or DCD) nanocluster adding to a LCD or LCL (DCD) surface nanocluster of the growing bowtie, respectively. The total surface area of the growing bowtie is then simply

$${A}_{T} = {P}_{d 1} {A}_{n 1} + ( {P}_{d 2} + {P}_{d 3} ) {A}_{n 2} \qquad ( 9 )$$

where P_{di} is the Boltzmann weighted growth probability in the i^{th} direction using the computed PMFs. Results from simulation suggests that when electrostatics repulsion dominates over H-bonding, the bowtie particles cease to grow, creating a self-limiting size effect. Here, it is assumed that each nanocluster gains 4 hydrogen bond upon incorporation into the growing bowtie. Additionally, the Cd²⁺ moieties within each nanocluster also contributes to local coordination as well as repulsion with other Cd²⁺ moieties on neighboring nanoclusters. These effects are incorporated into the computed PMFs (P_{di}) employed in Eq. 9. The size of the self-limiting assembly can be explicitly computed by equating the electrostatic repulsion with attraction from short-range forces. In other words, E_{Hbond}˜E_{charge}. Hydrogen bonds are generally on the order of KT, as such E_{Hbond} is taken to be of order unity (E_{Hbond}˜1). E_{charge} is approximated to be the total surface charge Q of the bowtie acting on the closest point on the surface of the growing front relative to the center of the bowtie. The distance to this proximal surface point is defined as R, allowing E_{charge}˜Q/R. When E_{Hbond}˜E_{charge}, this distance R becomes the self-limiting size of the particle R*. The total surface charge is simply the charge of the composite cluster distributed over the total surface area of the growing bowtie:

$$Q ∼ {nqA}_{T}^{- 1} .$$

such,

$$1 ∼ ( {nA}_{T}^{- 1} ) {qR}^{- 1} ,$$

where q is the charge of a nanocluster (taken to be of order unity). Rearranging for R* and taking the limit of A_{T} for n>>1 to reflect experimental observations that bowtie dimensions are orders of magnitude large than their constitutive building blocks result in Eq. 10.

$${R}^{*} ∼ {nA}_{T}^{- 1} ∼ n {\{ {P}_{1} [ {P}_{d 1} {γ}_{2} + ( {P}_{d 2} + {P}_{d 3} ) {γ}_{1}^{2} ] + {P}_{2} + α ( 2 {P}_{d 1} + {P}_{d 2} + {P}_{d 3} ) ( {P}_{1} {γ}_{1} {γ}_{2} + {P}_{2} ) + {P}_{1} [ {{P}_{d 1} ( {γ}_{2} - 1 )}^{2} + α ( {P}_{d 2} + {P}_{d 3} ) ( {γ}_{1} - 1 ) ] \}}^{- 1} \qquad ( 10 )$$

Eq. 10 defines the shortest radial distance from the center to the bowtie's surface. Therefore, the thickness of the bowtie is Rₜ˜R* [symbol] cos θ [symbol] . Similarly, the width is R_{w}˜R* [symbol] sin θ [symbol] ⁻¹ and length is Rₗ˜R_{w} [symbol] sin θ [symbol] ⁻¹. Comparison of theoretical prediction with both experiments and simulations yields results. Lastly, the number of nanoclusters n* in the self-limiting bowtie via ∂R*/∂n=0 can be explicitly determined by solving for n. Doing so gives

$${n}^{*} ∼ \{ {Γ}_{1} + {Γ}_{2} \} {\{ {\partial }_{n} {A}_{T , 1} + {\partial }_{n} {A}_{T , 2} \}}^{- 1} \qquad ( 11 )$$

$$where$$

$${Γ}_{1} = {P}_{d 1} \{ 1 - {P}_{1} [ {γ}_{1} + {( {γ}_{2} - 1 )}^{2} ] + {P}_{2} + 2 α [ 1 - {P}_{1} {γ}_{1} {γ}_{2} - {P}_{2} ] \}$$

$${Γ}_{2} = \{ {P}_{d 2} + {P}_{d 3} \} \{ 1 - {P}_{1} {γ}_{2}^{2} - {P}_{2} - α {P}_{1} {γ}_{1} \}$$

$${\partial }_{n} {A}_{T , 1} = {P}_{d 1} [ 2 α ( {P}_{1} {γ}_{1} {γ}_{2} + {P}_{2} ) + {P}_{1} ( {γ}_{2} - 1 ) + {P}_{1} {γ}_{2} {P}_{2} ]$$

$${\partial }_{n} {A}_{T , 2} = \{ {P}_{d 2} + {P}_{d 3} \} \{ α [ {P}_{1} ( {γ}_{1} {γ}_{2} + {γ}_{1} - 1 ) + {P}_{2} ] + {P}_{1} {γ}_{1}^{2} + {P}_{2} \}$$

Extension to Incomplete Nanocluster Formation. The above derivations assumes that only perfect Cd₂CST₂ nanoclusters form during the assembly process. Here, the theoretical construction is extended to account for the possible formation of incomplete nanoclusters. More specifically, the existence of 3 types of nanoclusters—Cd₂CST₂, Cd₂CST₁, and Cd₁CST₁ are considered. First, the fraction of incomplete cluster in the system is defined as δ. Rather than having a single P_{LCD} to balance with the Boltzmann weighting (Eq. 1), there are 6 probabilities corresponding to the each of Cd₂CST₂, Cd₂CST₁, and Cd₁CST₁ for both LCL (DCD) and LCD formations. Similar to Eq. 1, the reference state is taken to be that of P_{LCL}, reducing the problem to 5 coupled equations of the following form

$${δ}^{2} g ( χ ) = {Z}^{- 1} {e}^{- βΔ {U}_{2}} \qquad ( 12 a )$$

$$δ g ( χ ) = {Z}^{- 1} {e}^{- βΔ {U}_{3}} \qquad ( 12 b )$$

$$f ( χ ) = {Z}^{- 1} {e}^{- βΔ {U}_{4}} \qquad ( 12 c )$$

$$δ f ( χ ) = {Z}^{- 1} {e}^{- βΔ {U}_{5}} \qquad ( 12 d )$$

$${δ}^{2} f ( χ ) = {Z}^{- 1} {e}^{- βΔ {U}_{6}} \qquad ( 12 e )$$

where ΔU₂, ΔU₃, ΔU₄, AUS, and ΔU₆ are the energy difference a perfect Cd₂CST₂ formation and Cd₁CST₁ (LCL), Cd₂CST₁ (LCL), Cd₂CST₂ (LCD), Cd₂CST₁ (LCD), and Cd₁CST₁ (LCD), respectively. The functions Z, ƒ(χ), and g(χ) are defined such that:

$$Z = 1 + {\sum }_{i = 2}^{6} {e}^{- βΔ {U}_{i}}$$

$$g ( χ ) = {( \frac{1 + χ}{2 + χ} ) [ \frac{3}{4} + \frac{1}{4} {( 2 δ + 1 )}^{2} ]}^{- 1}$$

$$f ( χ ) = {( 1 - \frac{1 + χ}{2 + χ} ) [ \frac{3}{4} + \frac{1}{4} {( 2 δ + 1 )}^{2} ]}^{- 1}$$

Solving Eq. 12 gives the following set of general solutions:

$$βΔ {U}_{i} = - ln ( [ 1 + {Z}_{i + 1} ] \frac{{x}_{i}}{1 - {x}_{i}} )$$

$${x}_{2} = {δ}^{2} g ( χ ) \qquad ( 13 a )$$

$${x}_{3} = \frac{1}{δ} \frac{{x}_{2}}{1 - {x}_{2}} \qquad ( 13 b )$$

$${x}_{4} = f ( χ ) ( δ {x}_{3} + 1 ) ( 1 - \frac{{x}_{3}}{1 - {x}_{3}} ) \qquad ( 13 c )$$

$${x}_{5} = δ {x}_{4} ( 1 + \frac{{x}_{4}}{1 - {x}_{4}} ) \qquad ( 13 d )$$

$${x}_{6} = δ {x}_{5} ( 1 + \frac{{x}_{5}}{1 - {x}_{5}} ) \qquad ( 13 e )$$

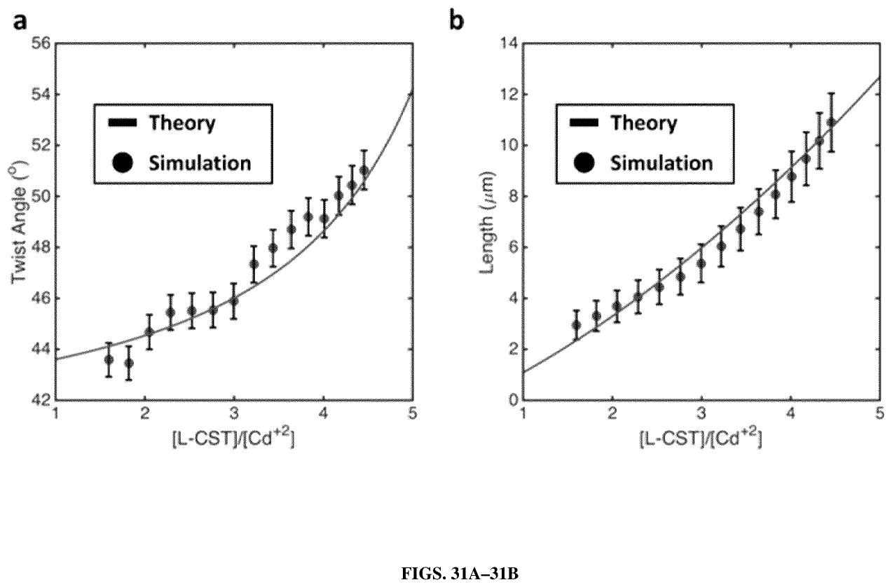

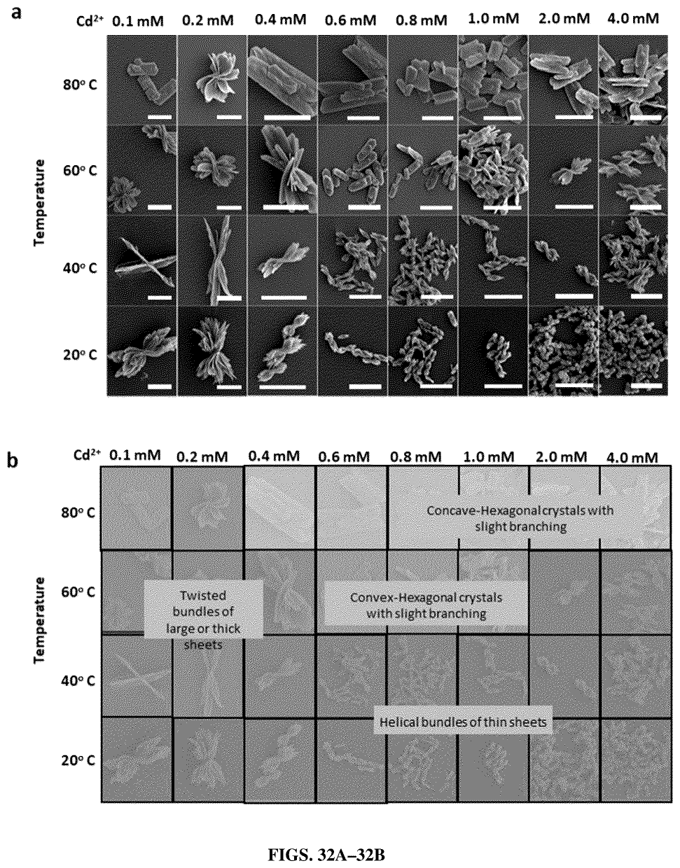

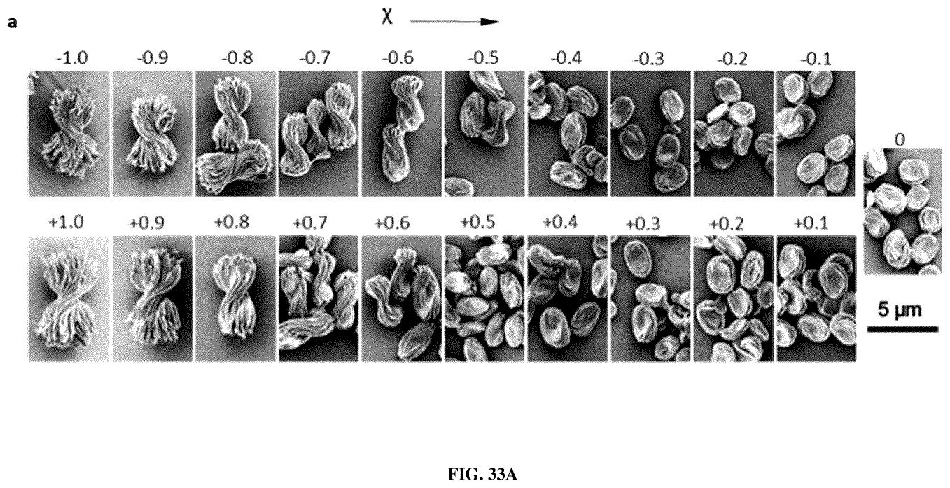

Eq. 13 can now be plugged into the governing equation for the twist angle (Eq. 2), but now the sum is over 6 enantiomeric states instead of 3. Solution for the twist angle directly plugs into subsequently equations to predict the dimensions of bowties outline in the previous section. Comparison of Monte Carlo growth simulation results incorporating the incomplete clusters with the theoretical extensions are shown in FIGS. 31A-31B (δ is mapped to experimental Cys/Cd ratio).

### Osipov-Pickup-Dunmur Chirality Measure (OPD) Calculations

Bowties parameters (length, width, thickness, twist angle) were extracted from the experimental data. Osipov-Pickup-Dunmur chirality measure (OPD) was calculated using the previously described method that uses the eight secondary center of masses extracted from different bowtie 3D models built with geometric parameters presented above. The center of each bowtie, which will be referred to as the primary center of mass, was placed at the (0,0,0) point of the Cartesian coordinate system. Bowtie length was aligned along x-axis and width along y-axis. Then, the bowtie was sectioned into eight pieces according to octants of the Cartesian system. Coordinates from their center of masses of the eight sections, referred to as secondary centers of masses, have been calculated. Using the coordinates of the eight secondary center of masses, OPD for all of the structures above were calculated.

The tensor gives rise to two universal chirality indices; the first giving information about absolute chirality, and the second about the anisotropy chirality, i.e., the degree of chirality in different spatial directions. The pseudoscalar behavior of chiral molecule can be described by the gyration tensor G,

$$G = \int {dr}_{1} {dr}_{2} {dr}_{3} {dr}_{4} ρ ( {r}_{1} ) ρ ( {r}_{2} ) ρ ( {r}_{3} ) ρ ( {r}_{4} ) \times \frac{[ ( {r}_{12} \times {r}_{34} ) ⊗ {r}_{14} ] ( {r}_{12} \cdot {r}_{23} ) ( {r}_{23} \cdot {r}_{34} )}{{( {r}_{12} {r}_{23} {r}_{34} )}^{n} {r}_{14}^{m}} ,$$

where rᵢⱼ=rᵢ−rⱼ and \hat{r}ᵢⱼ=rᵢⱼ/rᵢⱼ and n and m are arbitrary integers and ρ stands for the density of concentration. A scaled chiral index G_{0S} is a summation of contributions of all sets of four atoms in a numerical evaluation given by

$${G}_{0 S} = \frac{4 !}{{N}^{4}} \frac{1}{3} [ {\sum }_{\begin{matrix}all permutations of \\ i , j , k , l = 1 … N\end{matrix}} {w}_{i} {w}_{j} {w}_{k} {w}_{l} {G}_{0 S} = \frac{4 !}{{N}^{4}} {G}_{0} , \times \frac{[ ( {r}_{ij} \times {r}_{kl} ) \cdot {r}_{il} ] ( {r}_{ij} \cdot {r}_{jk} ) ( {r}_{jk} \cdot {r}_{kl} )}{{( {r}_{ij} {r}_{jk} {r}_{kl} )}^{n} {r}_{il}^{m}} ] ,$$

where N is total number of atoms in molecule, w is atomic weight=1 for all molecules considered here to provide a measure of steric chirality. In this calculation, the scaled chiral index is defined by calculating summation of contributions of all sets of four points from nine coordinates (primary and secondary center of masses, Table 1 in FIG. 10) using in-house MATLAB codes for OPD and UCSF Chimera.

### Single Particle Scattering Measurements

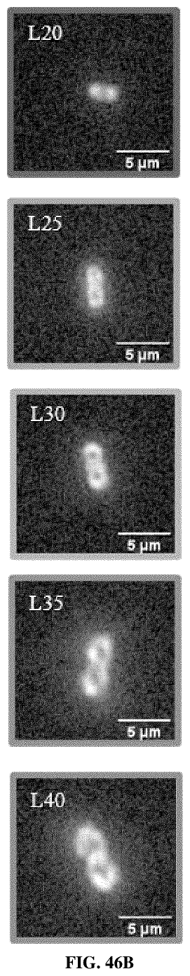

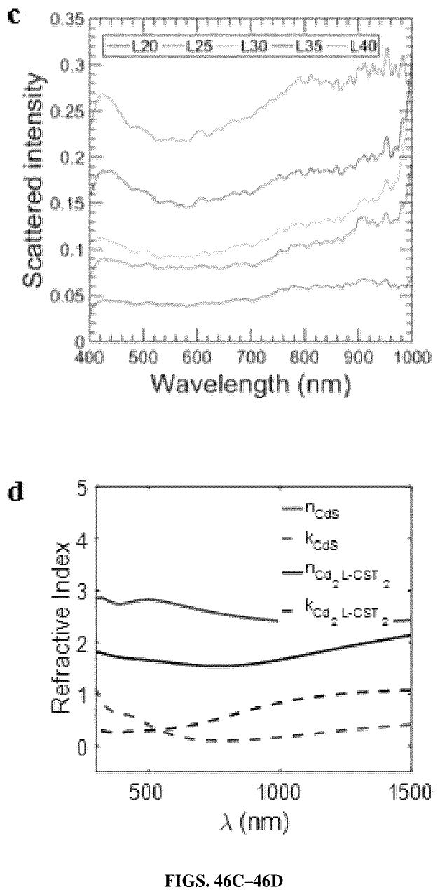

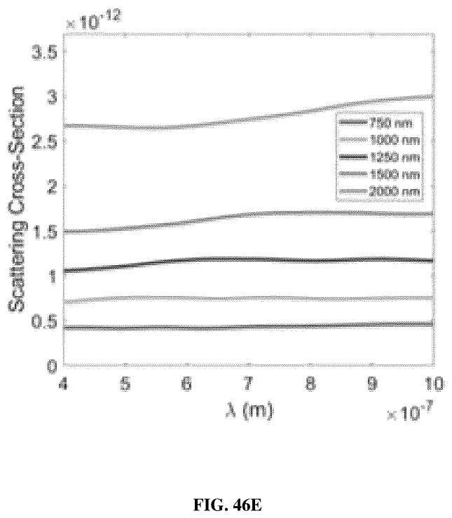

Transmission dark field scattering spectra are collected by a microspectrophotometer (CRAIC 308 PV) integrated on an optical microscope (Olympus BX51). White light from a 100 W tungsten halogen lamp is focused onto the sample drop casted on a glass substrate by using a substage dark field condenser (Olympus U-DCD), and dark field scattering from the sample is collected by a 40× objective (Olympus Plan N, NA 0.65). At 40× magnification, the collection aperture of the microspectrophotometer corresponds to a sample region of 6.9 μm×6.9 μm. The measured scattering intensity spectra is normalized by the lamp spectrum measured from a diffuse scattering reference (Labsphere) and using the expression I_{scat}=(Iₛₐₘₚₗₑ−I_{sample, background})/(I_{ref}−I_{ref, background}). See FIGS. 46a-46E for the corresponding data.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A chiral microparticle comprising:
an assembly comprising a plurality of nanoribbons that defines a bowtie shape and exhibits chirality.

2. The chiral microparticle of claim 1, wherein each nanoribbon of the plurality of nanoribbons comprises at least two peptides interconnected by at least one cadmium ion (Cd²⁺).

3. The chiral microparticle of claim 1, wherein each nanoribbon of the plurality of nanoribbons comprises at least two cystine molecules interconnected by at least one cadmium ion (Cd²⁺).

4. The chiral microparticle of claim 1, wherein each nanoribbon of the plurality of nanoribbons comprises at least two homocystine molecules interconnected by at least one cadmium ion (Cd²⁺).

5. The chiral microparticle of claim 1, wherein each nanoribbon of the plurality of nanoribbons has a length of greater than or equal to about 10 nm to less than or equal to about 100 micrometers.

6. The chiral microparticle of claim 1, wherein each nanoribbon of the plurality of nanoribbons has a thickness of greater than or equal to about 50 nm to less than or equal to about 10 micrometers.

7. The chiral microparticle of claim 1, wherein each nanoribbon of the plurality of nanoribbons has a width of greater than or equal to about 3 nm to less than or equal to about 20 micrometers.

8. The chiral microparticle of claim 1, wherein the chirality is within an OPD index of −150 to +150.

9. The chiral microparticle of claim 1, wherein the assembly defines the chiral microparticle having a length of greater than or equal to about 100 nm to less than or equal to about 100 micrometers.

10. The chiral microparticle of claim 1, wherein the assembly defines the chiral microparticle having a thickness of greater than or equal to about 500 nm to less than or equal to about 10 micrometers.

11. The chiral microparticle of claim 1, wherein the assembly defines the chiral microparticle having a width of greater than or equal to about 50 nm to less than or equal to about 30 micrometers.

12. The chiral microparticle of claim 1, wherein the assembly defines the chiral microparticle having a pitch of greater than or equal to about 10 nm to less than or equal to about 100 micrometers.

13. A chiral dispersion comprising:
a plurality of chiral microparticles each comprising a plurality of nanoribbons distributed in a medium, wherein at least one first chiral microparticle in the plurality of chiral microparticles exhibits a first chirality that is distinct from a second chirality exhibited by at least one second chiral microparticle in the plurality of chiral microparticles.

14. The chiral dispersion of claim 13, wherein the plurality of nanoribbons each comprises at least two peptides interconnected by at least one cadmium ion (Cd²⁺).

15. The chiral dispersion of claim 13, wherein the plurality of nanoribbons each comprises at least two cystine molecules interconnected by at least one cadmium ion (Cd²⁺).

16. The chiral dispersion of claim 13, wherein the plurality of nanoribbons each comprises at least two homocystine molecules interconnected by at least one cadmium ion (Cd²⁺).

17. The chiral dispersion of claim 13, wherein the plurality of nanoribbons each has a length of greater than or equal to about 10 nm to less than or equal to about 100 micrometers, a thickness of greater than or equal to about 50 nm to less than or equal to about 10 micrometers, and a width of greater than or equal to about 3 nm to less than or equal to about 20 micrometers.

18. The chiral dispersion of claim 13, wherein the first chirality is within an OPD index of −150 to +150.

19. The chiral dispersion of claim 13, wherein the at least one first chiral microparticle has a length of greater than or equal to about 100 nm to less than or equal to about 100 micrometers, a thickness of greater than or equal to about 500 nm to less than or equal to about 10 micrometers, a width of greater than or equal to about 50 nm to less than or equal to about 30 micrometers, and a pitch of greater than or equal to about 10 nm to less than or equal to about 100 micrometers.
